(19) United States
(12) Patent Application Publication
(10) Pub. No.: US 2022/0254467 A1
(43) Pub. Date: Aug. 11, 2022

(54) SYSTEM AND METHOD FOR ENHANCED COMMUNICATION BETWEEN A RETAIL PHARMACY ENTERPRISE AND A PATIENT

(71) Applicant: Meijer, Inc., Grand Rapids, MI (US)

(72) Inventor: Jeffrey Marentette, Grand Rapids, MI (US)

(21) Appl. No.: 17/592,124

(22) Filed: Feb. 3, 2022

Related U.S. Application Data
(60) Provisional application No. 63/146,771, filed on Feb. 8, 2021.

(51) Int. Cl. G16H 20/10; G16H 10/60; G06F 21/62; G06Q 30/06

(52) U.S. Cl. CPC G16H 20/10; G06Q 30/0607; G06F 21/6254; G16H 10/60

(57) ABSTRACT

A system and method for enhanced messaging with a patient of a retail pharmacy matches a data payload to one a plurality of communication rules, wherein each of the plurality of communication rules is associated with a message type, and wherein the data payload is indicative of a prescription transaction provided to the retail pharmacy for the patient. A message is created based on the message type associated with the matching one of the plurality of communication rules, and is sent to a mobile communication device associated with the payload and with the patient. The message includes an application address associated with the message type. Upon selection of the application address via patient interaction with the mobile communication device, a progressive web application associated with the application address is executed via which the retail pharmacy interactively provides at least one retail pharmacy service to the patient.

### CROSS-REFERENCE TO RELATED APPLICATION

This application claims the benefit of, and priority to, U.S. Provisional Patent Application Ser. No. 63/146,771, filed Feb. 8, 2021, the disclosure of which is expressly incorporated herein by reference in its entirety.

### FIELD OF THE INVENTION

The present invention relates generally to systems and methods for automatically communicating with customers of a retail pharmacy enterprise, and more specifically to such systems and methods for automatically identifying patients with pending prescription transactions and automatically generating enhanced messaging interactions.

### BACKGROUND

Retail pharmacies can be found in standalone locations, near hospital locations, or within large “big-box” retail stores. Typical patient transactions at retail pharmacies include in-person patient interactions such as receiving a patient signature, adding payment methods, and providing counseling regarding prescriptions. Retail pharmacies may allow patients to access prescription information and other data using mobile applications. Typical mobile applications require the patient to download a custom application prior to accessing information. Additionally, typical mobile applications may require the patient to set up an online account before accessing information.

### SUMMARY

The present invention may comprise one or more of the features recited in the attached claims, and/or one or more of the following features and combinations thereof. In a first aspect, a method for enhanced messaging with a patient of a retail pharmacy patient may comprise matching, by a cloud server, a data payload to a plurality of communication rules, wherein each of the plurality of communication rules is associated with a message type, and wherein the data payload is indicative of a prescription transaction provided to the retail pharmacy for the patient; creating, by the cloud server, a message based on the message type associated with the matching one of the plurality of communication rules, wherein the message includes an application address associated with the message type, sending, by the cloud server, the message to a mobile communication device associated with the data payload and with the patient, and, in response to receipt by the cloud server of selection of the application address via patient interaction with the mobile communication device, executing, by the cloud server with the mobile communication device, a progressive web application associated with the application address via which the retail pharmacy interactively provides at least one retail pharmacy service to the patient.

In a second aspect, a cloud server for enhanced messaging with a patient of a retail pharmacy may comprise a processor and a memory having instructions stored therein and executable by the processor to cause the processor to match a data payload to one of a plurality of communication rules, wherein each of the plurality of communication rules is associated with a message type, and wherein the data payload is indicative of a prescription transaction provided to the retail pharmacy for the patient, create a message based on the message type associated with the matching one of the plurality of communication rules, wherein the message includes an application address associated with the message type, send the message to a mobile communication device associated with the data payload and with the patient, and in response to receipt of selection of the application address via patient interaction with the mobile communication device, execute with the mobile communication device a progressive web application associated with the application address via which the retail pharmacy interactively provides at least one retail pharmacy service to the patient.

In a third aspect, a non-transitory machine-readable medium may comprise a plurality of instructions executable by at least one processor to cause the at least one processor to match a data payload to one of a plurality of communication rules, wherein each of the plurality of communication rules is associated with a message type, and wherein the data payload is indicative of a prescription transaction provided to the retail pharmacy for the patient, create a message based on the message type associated with the matching one of the plurality of communication rules, wherein the message includes an application address associated with the message type, send the message to a mobile communication device associated with the data payload and with the patient, and in response to receipt of selection of the application address via patient interaction with the mobile communication device, execute with the mobile communication device a progressive web application associated with the application address via which the retail pharmacy interactively provides at least one retail pharmacy service to the patient.

A fourth aspect may include the features of one or any combination of the first through third aspects and may further include sending, by the cloud server, a request for verifying information to the mobile communications device in response to selection of the application address via patient interaction with the mobile communication device, receiving, by the cloud server, a patient selection indicative of the verifying information from the mobile communications device in response to sending the request, and determining, by the cloud server, whether the patient selection matches an item of verifying information from the data payload. In some embodiments, verifying information may include a date of birth of the patient. In some embodiments, the verifying information may include a personal identification number (PIN).

A fifth aspect may include the features of any one or combination of the first through fourth aspects and may further include receiving, by the cloud server, the data payload from an enterprise pharmacy system, wherein matching the data payload may comprise matching the data payload in response to receiving the data payload.

A sixth aspect may include the features of any one or combination of the first through fifth aspects, wherein executing the progressive web application may include executing an express checkout shopping cart application via which the cloud server provides a prescription shopping cart interface to the mobile communication device for selection by the patient, via interaction with the mobile communication device, of one or more elements of a prescription transaction including a payment method. In some embodiments, executing the express checkout shopping cart application may include determining whether the data payload includes a user selection to opt in to view details, and masking private health information in response to determining that the data payload does not include the user selection to opt in. In some embodiments, executing the express checkout shopping cart application may include updating a payment method, capturing a patient signature and/or capturing a patient survey response. In some embodiments, executing the express checkout shopping cart application may include generating a barcode indicative of the prescription transaction, and transmitting the barcode to the mobile communications device.

A seventh aspect may include the features of any one or combination of the first through six aspects, wherein executing the progressive web application may alternatively or additionally include executing a view details enrollment application via which the patient provides patient information to the cloud server by interacting with the mobile communication device. In some embodiments, executing the progressive web application may alternatively or additionally include executing a drug recall application via which the cloud server sends at least one message to the mobile communication device informing the patient of drug recall information. In some embodiments, executing the progressive web application may alternatively or additionally include executing a prescription refill application via which the cloud server provides a prescription refill interface to the mobile communication device for selection by the patient, via interaction with the mobile communication device, of one or more elements of a prescription refill request. In some embodiments, executing the progressive web application may alternatively or additionally include executing an immunization application via which the cloud server provides an immunization request interface to the mobile communication device for selection by the patient, via interaction with the mobile communication device, of one or more elements for scheduling an immunization appointment with the retail pharmacy.

An eighth aspect may include the features of any one or combination of the first through seventh aspects, and may further include generating, by the cloud server, a progressive web application interface for the progressive web application from a plurality of application templates based on the data payload and in response to selection of the application address via patient interaction with the mobile communication device, and transmitting, by the cloud server, the progressive web application interface to the mobile communications device. Some embodiment may further include receiving, by the cloud server, a patient selection from the mobile communications device in response to transmitting the progressive web application interface, and updating, by the cloud server, the data payload based on the patient selection.

In some embodiments of any or combination of the first through eighth aspects, a second message may be received, by the cloud server from the first mobile communications device, wherein creating the message comprises creating a first message in response to receiving the second message. Alternatively or additionally, some embodiments may further include determining, by the cloud server, a progressive web application interaction component based on the data payload in response to receipt by the cloud server of selection of the application address, generating, by the cloud server, a or the progressive web application interface from a plurality of application templates in response to determining the progressive web application interaction component, and transmitting, by the cloud server, the progressive web application interface to the mobile communications device. In some embodiments, determining the progressive web application interaction component may include identifying the progressive web application interaction component based on a predetermined configuration table of the cloud server. In some embodiments, the progressive web application interaction component may comprise a verify patient identity component, a read message component, a question component, a survey component, a review documents component, a signature capture component, and/or a complete screen component.

### BRIEF DESCRIPTION OF THE DRAWINGS

This disclosure is illustrated by way of example and not by way of limitation in the accompanying figures. Where considered appropriate, reference labels have been repeated among the figures to indicate corresponding or analogous elements.

FIG. 1 is a simplified block diagram of an embodiment of a system for enhanced patient communication for a retail pharmacy enterprise.

FIG. 2 is a simplified block diagram of an embodiment of a software environment of the main server of in FIG. 1.

FIG. 3 is a simplified block diagram of an embodiment of a software environment of the cloud server of in FIG. 1.

FIG. 4 is a simplified block diagram of an embodiment of a software environment of one of the mobile communication devices of in FIG. 1.

FIG. 5 is a simplified flow diagram of an embodiment of a process for enhanced messaging that may be executed by the system of FIG. 1.

FIG. 6 is a simplified flow diagram of an embodiment of a process for express checkout with enhanced messaging.

FIG. 7 is a simplified flow diagram of an embodiment of a process for identify verification that may be executed as part of the process illustrated in the flow diagram of FIG. 6.

FIG. 8 is a simplified flow diagram of an embodiment of a process for express checkout shopping cart management that may be executed as part of the process illustrated in the flow diagram of FIG. 6.

FIG. 9 is a simplified flow diagram of an embodiment of a process for enhanced messaging enrollment.

FIG. 10 is a simplified flow diagram of an embodiment of a process for prescription ready time modification with enhanced messaging.

FIG. 11 is a simplified flow diagram of an embodiment of a process for drug recall notification with enhanced messaging.

FIG. 12 is a simplified flow diagram of an embodiment of a process for a prescription refill reminder with enhanced messaging.

FIG. 13 is a simplified flow diagram of an embodiment of a process for a prescription refill request with enhanced messaging.

FIG. 14 is a simplified flow diagram of another embodiment of a process for identify verification.

FIG. 15 is a simplified flow diagram of an embodiment of a process for immunization request processing with enhanced messaging.

FIG. 16 is a simplified flow diagram of an embodiment of a process for immunization appointment scheduling with enhanced messaging.

FIG. 17 is a simplified flow diagram of an embodiment of a process for dynamic two-way communication with enhanced messaging.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases may or may not necessarily refer to the same embodiment. Further, when a particular feature, structure, process, process step or characteristic is described in connection with an embodiment, it is submitted that it is within the knowledge of one skilled in the art to effect such feature, structure, process, process step or characteristic in connection with other embodiments whether or not explicitly described. Further still, it is contemplated that any single feature, structure, process, process step or characteristic disclosed herein may be combined with any one or more other disclosed feature, structure, process, process step or characteristic, whether or not explicitly described, and that no limitations on the types and/or number of such combinations should therefore be inferred.

Embodiments of the invention may be implemented in hardware, firmware, software, or any combination thereof. Embodiments of the invention implemented in a computer system may include one or more bus-based interconnects between components and/or one or more point-to-point interconnects between components. Embodiments of the invention may also be implemented as instructions stored on one or more machine-readable media, which may be read and executed by one or more processors. A machine-readable medium may be embodied as any device or physical structure for storing or transmitting information in a form readable by a machine (e.g., a computing device). For example, a machine-readable medium may be embodied as any one or combination of read only memory (ROM); random access memory (RAM); magnetic disk storage media; optical storage media; flash memory devices; and others.

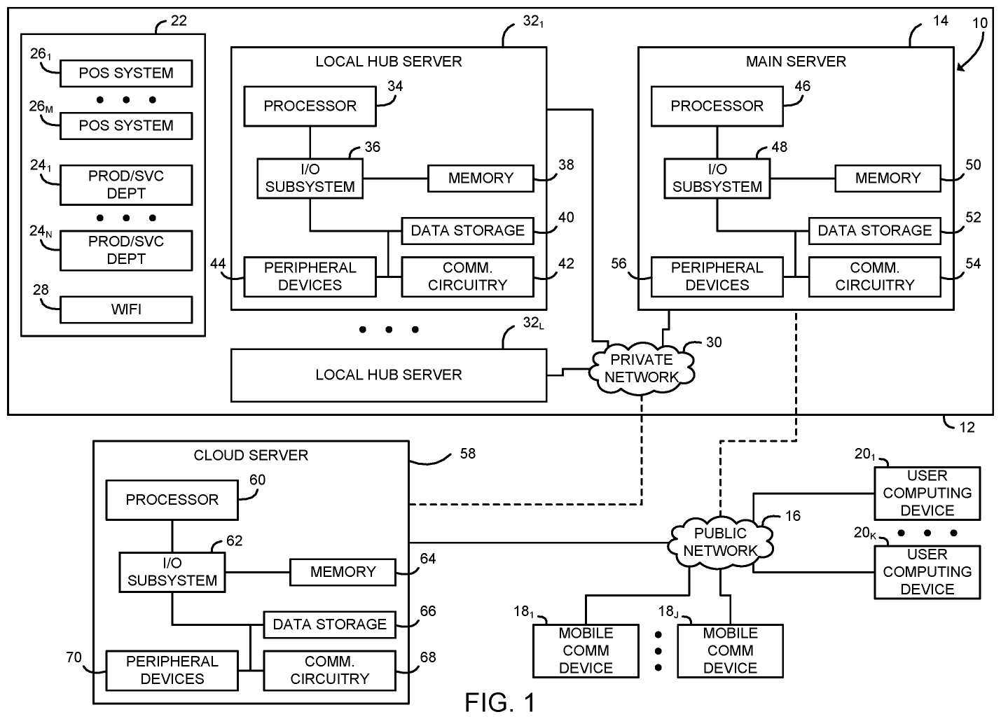

Referring now to FIG. 1, a system 10 is shown for enhanced patient communication with a retail pharmacy enterprise. The system 10 includes a retail pharmacy enterprise 12 having a main server 14 configured to communicate with patients and/or other shoppers via a public network 16, e.g., the Internet, and patients may access the public network 16 using any conventional public network accessible electronic device and/or system. In the illustrated embodiment, for example a number, J, of mobile communication devices 18₁-18_{J}, and a number, K, of user computing devices 20₁-20_{K}, are shown. Each is configured to communicatively connect to the public network 16, and J and K may each be any positive integer.

The illustrative retail pharmacy enterprise 12 is part of a retail enterprise sometimes referred to as a “Big-Box Store,” “Superstore,” “Supercenter,” or “Megastore,” having multiple external retail outlets or stores 22, each of which include therein multiple product/service departments 24. Examples of such product/service departments 24 include, but are not limited to, the pharmacy, a bakery, a meat department, seafood department, a dairy department, a produce department, a beverage department, a frozen food department, a photograph developing service department, an electronics department, a sporting goods department, a nursery, a seasonal goods department, a clothing department, a shoe department, a pet food and/or accessory department, an automotive goods department, and kitchenware department, a hardware department, and the like. Additionally or alternatively, in some embodiments the illustrative retail pharmacy enterprise 12 may include multiple external retail pharmacies 22 that are not included in a larger retail outlet. In still other embodiments, the retail pharmacy 22 may be a sole or stand-alone retail pharmacy, i.e., not part of a larger retail pharmacy enterprise or other retail enterprise.

As discussed above, the retail pharmacy enterprise 12 may include any number of brick-and-mortar retail outlets each having one or more point-of-sale systems 26₁-26_{M} operating therein. The main server 14 is configured to communicate with each such point-of-sale (POS) system 26₁-26_{M}, each of which operate in a conventional manner to process items to be purchased by shoppers during purchase transactions.

Each of the brick-and-mortar stores 22 may further include at least one conventional WiFi Access Point 28 which may be coupled to the corresponding local hub server 32, or directly to the main server 14 in any one or more of the brick-and-mortar stores 22 not having an associated local hub server 32. Each such WiFi Access Point 28 is illustratively controlled by the main server 14 (or corresponding local hub server 32) in a conventional manner to establish at least one corresponding Internet hotspot within the brick-and-mortar store 22 via which customers (and employees) can access the public network 16, e.g., to access the Internet, using any conventional public network accessible electronic device and/or system, e.g., such as with any of the plurality of mobile communication devices 18₁-18_{J}.

In some embodiments, the main server 14 illustratively hosts an enterprise member or membership services (EMS) program which includes or otherwise has access to a virtual coupon bank and a customer purchase history database containing purchase histories of one or more customers of the retail pharmacy enterprise 12. As used herein, the terms “enterprise member services program,” “EMS program” and “customer membership service” are interchangeable and refer to a shopper or customer service which a retail pharmacy enterprise 12 may offer to customer members in the form of one or more services such as making available to customers one or more virtual discount coupons that may be redeemable by the retail enterprise against the purchase of from the retail enterprise of various goods and/or services and/or tracking and maintaining customer purchase histories in a customer purchase history database accessible by the main server 14. In this regard, the terms “customer membership account” and “EMS account” are likewise interchangeable and refer to a mechanism by which the retail pharmacy enterprise 12 may make available to customers one or more virtual discount coupons and/or by which a customer's purchase history and information about the customer can be maintained by the main server 14 in a database separately from purchase histories of and information about other customers. Further in this regard, the term “EMS identification code” or EMSID illustratively refers to at least one collection of letters, symbols and/or numbers that is different for, and therefore unique to, each customer member of the enterprise membership services program, and which is used to uniquely identify a customer's EMS account within the enterprise membership services program. In one embodiment, for example, the EMSID for each customer may include a unique, several-digit access code and a separate and unique, several-digit password, although in other embodiments the EMSID may include more, fewer and/or different codes and/or passwords.

The main server 14 may include an EMS module that manages and controls a customer-member interface, e.g., a web-based interface, to the EMS program via which customers can access and manage their individual EMS accounts. Illustratively, each customer may access their individual (and private from other customer-members) EMS account, i.e., their individual EMS page(s) within the web-based EMS interface, which may be referred to hereinafter as an “EMS website,” by entering that customer's EMSID into a graphic user interface element of the web-based EMS interface. Therein, the customer may access, establish, modify and otherwise manage the customer's EMS account information including, for example, but not limited to, name, address, email address, mobile telephone number and the like.

In the embodiment illustrated in FIG. 1, the main server 14 is coupled via a private network 30 to a plurality of local hub servers 32₁-32_{L}, where L may be any positive integer, and each local hub server 32₁-32_{L} is coupled to one or more conventional point-of-sale systems, e.g., 26₁-26_{M}. Each of the point-of-sale systems 26₁-26_{M} is located in a different one of one or more brick-and-mortar stores of the retail pharmacy enterprise 12, and is configured to process items selected by customers for purchase at a corresponding brick-and-mortar store. While only one such brick-and-mortar store 22 is shown in FIG. 1, it will be understood that each of the local hub servers 32₁-32_{L} may be coupled to a different brick-and-mortar store, each as illustrated with respect to the brick-and-mortar store 22, such that the retail pharmacy enterprise 12 may include any number, L, of brick-and-mortar stores 22₁-22_{L}. Some retail enterprises 11 may include a single brick and mortar store, and other larger enterprises may include two or more physically remote brick and mortar stores. In the latter case, the retail pharmacy enterprise 12 may include, for example, a main physical location with two or more remote physical locations, and for purposes of this document the two or remote physical locations in such an arrangement are referred to as “hub” locations. In this disclosure, the system 10 will be illustrated and described in the context of such a larger retail enterprise having a main physical location and two or more physical hub locations. In this regard, the main server 14 in the system 10 shown in FIG. 1 will typically be located at a main business location of the retail enterprise, and will be coupled via the network 30 to two or more local hub servers 32₁-32_{L}, each of which will typically be located at a different one of the two or more hub locations.

Each hub location may include any number of point-of-sale systems coupled to a corresponding local hub server, and in the embodiment illustrated in FIG. 1, for example, the local hub server 32₁ is communicatively coupled to “M” such point-of-sale systems 26₁-26_{M}, where M may be any positive integer. Communicative coupling between the local hub server 32₁ and the one or more point-of-sale systems 26₁-26_{M} may be accomplished using any known communication coupling, and communications over any such hardwire and/or wireless coupling may be accomplished using any known communication protocol.

In some alternative embodiments of such a large retail enterprise, one or more of the local hub servers 32₁-32_{L} may be omitted, and the main server 14 may be coupled directly, via the network 30, to one or more point-of-sale systems 26₁-26_{M} or the main server 14 may be omitted and at least one of the local hub servers 32₁-32_{L} may be configured to act as a so-called master server with the remaining local hub servers 32₁-32_{L} configured to act as so-called slave servers. In other alternative embodiments in which the retail enterprise includes only a single brick and mortar store, the local hub servers 32₁-32_{L} may be or include the main server 14 or vice versa. For purposes of the following description, any process disclosed as being controlled by the main server 14 may, in some embodiments, instead be controlled, in whole or in part, by one or more local hub servers 32₁-32_{L} and vice versa, and/or may be controlled, in whole or in part, by one of the point-of-sale systems 26₁-26_{M} and vice versa.

The local hub server 32₁ may be embodied as any type of server or similar computing device capable of performing the functions described herein. In the illustrative embodiment of FIG. 1, the local hub server 32₁ includes a processor 34, an I/O subsystem 36, a memory 38, a data storage 40, a communication circuitry 42, and one or more peripheral devices 42. It should be appreciated that the local hub server 32₁ may include other components, sub-components, and devices commonly found in a server and/or computing device, which are not illustrated in FIG. 1 for clarity of the description.

The processor 34 of the local hub server 32₁ may be embodied as any type of processor capable of executing software/firmware, such as a microprocessor, digital signal processor, microcontroller, or the like. The processor 34 may be a single processor or include multiple processors. The I/O subsystem 32 of the local hub server 32₁ may be embodied as circuitry and/or components to facilitate input/output operations with the processor 34 and/or other components of the local hub server 32₁. The processor 34 is communicatively coupled to the I/O subsystem 36.

The memory 38 of the user local hub server 104 may be embodied as or otherwise include one or more conventional volatile and/or non-volatile memory devices. The memory 38 is communicatively coupled to the I/O subsystem 36 via a number of signal paths. Although only a single memory device 38 is illustrated in FIG. 1, the local hub server 32₁ may include additional memory devices in other embodiments. Various data and software may be stored in the memory 38. The data storage 40 is also communicatively coupled to the I/O subsystem 36 via a number of signal paths, and may be embodied as any type of device or devices configured for the short-term or long-term storage of data such as, for example, memory devices and circuits, memory cards, hard disk drives, solid-state drives, or other data storage devices.

The communication circuitry 42 of the local hub server 32₁ may include any number of devices and circuitry for enabling communications between the local hub server 32₁ and the main server 14 and between the local hub server 32₁ and the one or more point-of-sale systems 26₁-26_{M}. In the illustrated embodiment, for example, communication between the local hub server 32₁ and the main server 14 takes place wirelessly via the network 30, wherein the network 30 may represent, for example, a private local area network (LAN), personal area network (PAN), storage area network (SAN), backbone network, global area network (GAN), wide area network (WAN), or collection of any such computer networks such as an intranet, extranet or the Internet (i.e., a global system of interconnected network upon which various applications or service run including, for example, the World Wide Web). In alternative embodiments, the communication path between the local hub server 32₁ and the main server 14 may be a non-private network and/or may be, in whole or in part, a wired connection. Generally, the communication circuitry 42 may be configured to use any one or more, or combination, of conventional secure and/or unsecure communication protocols to communicate with the main server 14. As such, the network 30 may include any number of additional devices, such as additional computers, routers, and switches, to facilitate communications between the local hub server 32₁ and the main server 14. Communication between the local hub server 32₁ and the one or more point-of-sale systems 26₁-26_{M} may take place via one or more such wireless communication interfaces and/or via one or more conventional wired interfaces.

In some embodiments, the local hub server 32₁ may also include one or more peripheral devices 42. Such peripheral devices 40 may include any number of additional input/output devices, interface devices, and/or other peripheral devices. For example, the peripheral devices 42 may include a display, a keyboard, a mouse, audio processing circuitry, and/or other input/output devices.

The local hub server 32_{L} may be substantially similar to the local hub server 32₁ and include similar components. As such, the description provided above of the components of the local hub server 32₁ may be equally applicable to such similar components of the local hub server 32_{L} and are not repeated herein so as not to obscure the present disclosure. Of course, it should be appreciated that in some embodiments one or more of the local hub servers 32₁-32_{L} and may be dissimilar to others of the local hub servers 32₁-32_{L}.

An embodiment of the main server 14 is also illustrated in FIG. 1, and generally includes the same components as the local hub server 32₁. For example, a processor 46 is coupled to an I/O subsystem 48, and the I/O subsystem 48 is coupled to a memory 50, a data storage unit 52, communication circuitry 54 and one or more peripheral devices 56. In some embodiments, each of the foregoing components may be identical to corresponding components of the local hub server 32₁ described above, and a detailed explanation of such components will not be repeated here for brevity. In other embodiments, the main server 14 may be configured differently than the local hub server 32₁ described above. In any case, the communication circuitry 42 of each of the local hub servers 32₁-32_{L} facilitates communication with the communication circuitry 54 of the main server 14 and vice versa so that information can be shared between the main server 14 and each of the one or more local hub servers 32₁-32_{L} via the network 30. Although only one such main server 14 is shown in FIG. 1, it should be appreciated that, in other embodiments, the system 10 may include any number of main servers, and in still other embodiments the main server 14 may be communicatively coupled to one or more remote servers of the retail enterprise. In such embodiments, the one or more remote servers may include any structure or feature illustrated and described herein with respect to the main server 14, and may be configured to execute any one or more functions described with respect to the main server 14 either alternatively to the main server 14 or in addition to the main server 14. In any case, the main server 14 may be embodied as any type of server or similar computing device capable of performing the functions described herein.

The mobile communication devices 18₁-18_{J} illustrated in FIG. 1 are intended to depict mobile communication devices that are each separately owned and/or operated by a different patient and/or other shopper. No limit on the total number of such mobile communication devices 18₁-18_{J} that may be owned and operated by any one shopper, or on the total number of such mobile communication devices 18₁-18_{J} that may communicate with the main server 14, is intended or should be inferred. The mobile communication devices 18₁-18_{J} may be or include any mobile electronic device capable of executing one or more software application programs as described herein and of communicating with the main server 14 via the public network 16. Examples of the mobile communication devices 18₁-18_{J} include, but should not be limited to, mobile telephones, smart phones, tablet computers, personal data assistants (PDAs), and the like.

The user computing devices 20₁-20_{K} illustrated in FIG. 1 are intended to include any of privately owned and accessed computers, such as those residing in customer's residences, to include semi-privately owned and accessed computers, such as those residing at multiple-employee business enterprises, and publicly accessible computers, such as those available at internet café s and kiosks. The user computing devices 20₁-20_{K} may be or include any computer capable of executing one or more software programs and of communicating with the main server 14 via the public network 16 for various purposes including, for example, accessing by customers of their EMS web page(s). Examples of the user computing devices 20₁-20_{K} include, but should not be limited to, personal computers (PCs), laptop computers, notebook computers and the like, whether or not networked with one or more other computing devices.

Also depicted in FIG. 1 are a number of product/service departments 24₁-24_{N} each illustratively coupled to the local hub server 32 in a different one of the brick-and-mortar enterprise locations 22₁-22_{L} such that each brick-and-mortar enterprise location includes a one or more such product/service departments 24₁-24_{N}. In alternate embodiments, one or more or all of the product/service departments 24₁-24_{N} may not be coupled to the local hub server 32 but may instead be coupled directly, e.g., via the private network 30, to the main server 14. As described above, the product/service departments 24₁-24_{N} each illustratively comprise a separate department within each brick-and-mortar store, and each such product/service department illustratively offers a particular product or service, or a particular set of products or services, typically not offered by any other department within the same brick-and-mortar store. One or more of the product/service departments 24₁-24_{N} may include an electronic system or device located therein or otherwise associated therewith.

As shown in FIG. 1, the main server 14 and the mobile communication devices 18₁-18_{J} are coupled via the public network 16 to a cloud server 58. As illustrated in FIG. 1, in some embodiments the cloud server 58 may also access the private network 30, for example via one or more virtual private network connections or other private network connections. The cloud server 58 may be embodied as, without limitation, any server or other computing device capable of performing the functions described herein, and thus generally includes the same components as the local hub server 32₁ and/or the main server 14. For example, a processor 60 is coupled to an I/O subsystem 62, and the I/O subsystem 62 is coupled to a memory 64, a data storage unit 66, communication circuitry 68, and one or more peripheral devices 70. In some embodiments, each of the foregoing components may be identical to corresponding components of the local hub server 32₁ described above, and a detailed explanation of such components will not be repeated here for brevity. In other embodiments, the main server 14 may be configured differently than the local hub server 32₁ described above. Additionally, in some embodiments, the cloud server 58 may be embodied as a “virtual server” formed from multiple computing devices distributed across the network 16 and operating in a public or private cloud. Accordingly, although the cloud server 58 is illustrated in FIG. 1 as embodied as a single computing device, it should be appreciated that the cloud server 58 may be embodied as multiple devices cooperating together to facilitate the functionality described below.

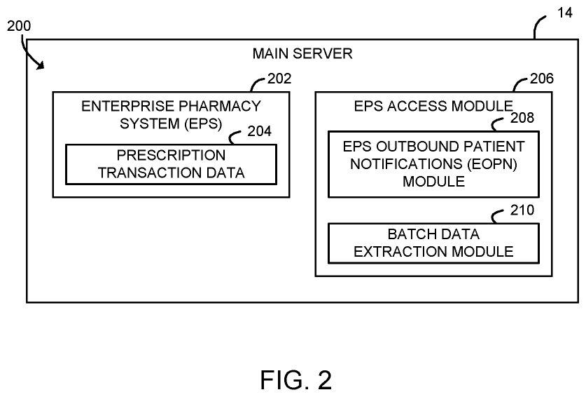

Referring now to FIG. 2, a simplified block diagram is shown of an embodiment of an environment 200 of the main server 14 illustrated in FIG. 1. In the embodiment shown in FIG. 2, the environment 200 includes an enterprise pharmacy system (EPS) 202, which illustratively includes prescription transaction data 204.

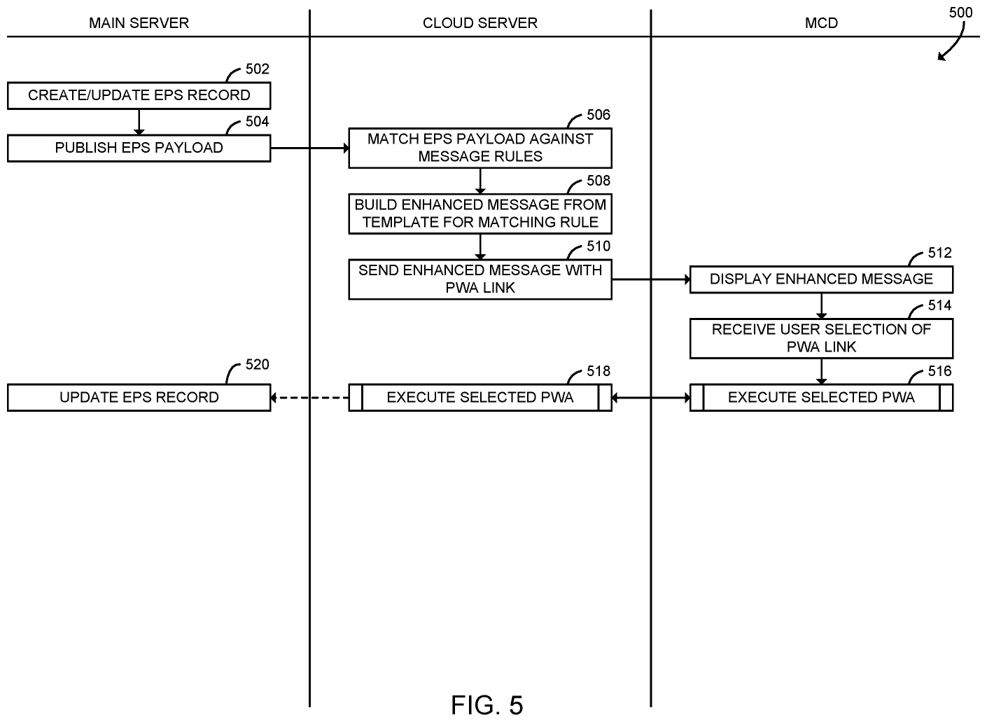

The environment 200 of the main server 14 further includes an EPS access module 206, which illustratively includes an EPS outbound patient notifications (EOPN) module 208 and a batch data extraction module 210. The EPS access module 206 is illustratively operable to manage access to the EPS 202 performed by the cloud server 58. In some embodiments, the EOPN module 208 is operable to publish EPS 202 data payloads to the cloud server 58. In some embodiments, the batch data extraction module 210 is operable to extract and batch load EPS 202 data, such as drug recall data or prescription refill data. Example embodiments of processes executed by the EPS access module 206 are illustrated in FIGS. 5-6, 9-13, and 16-17 and such processes will be described in detail hereinafter.

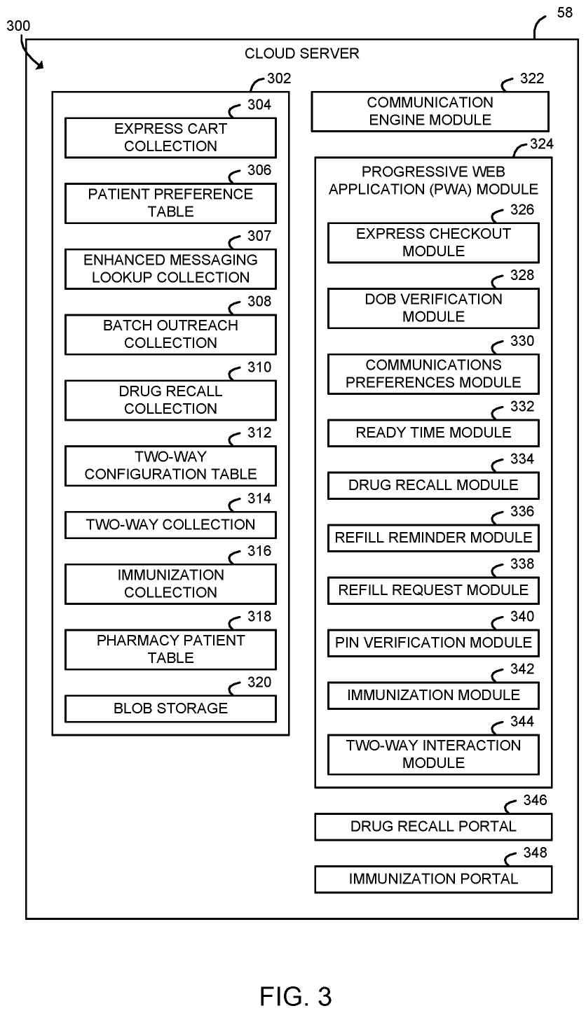

Referring now to FIG. 3, a simplified block diagram is shown of an embodiment of an environment 300 of the cloud server 58 illustrated in FIG. 1. In the embodiment shown in FIG. 3, the environment 300 includes multiple data stores 302, which may be embodied as one or more databases, data tables, data collections, files, bulk storage, or other data storage maintained or otherwise accessible by the cloud server 58. Illustratively, the data stores 302 include an express cart collection 304, a patient preference table 306, an enhanced messaging lookup collection 307, a batch outreach collection 308, a drug recall collection 310, a two-way configuration table 312, a two-way collection 314, an immunization collection 316, a pharmacy patient table 318, and blob storage 320.

The environment 300 of the cloud server 200 further includes a communication engine module 322, a progressive web application (PWA) module 324, a drug recall portal 346, and an immunization portal 348. The communication engine module 322 is illustratively operable to evaluate one or more communication rules and perform enhanced messaging with one or more mobile communications devices (MCDs) 18 that are operated by pharmacy patients of the pharmacy enterprise 12. The PWA module 324 is illustratively operable to provide one or more PWA interfaces to the MCDs 18. The term “progressive web application” or “PWA” is to be understood in accordance with its ordinary meaning; that is, a progressive web application (PWA) is a website that looks and behaves, with respect to a mobile communication device such as an MCD 18, as if it is a native mobile application running on the mobile device. PWAs generally take advantage of native mobile device features, without requiring the mobile device user to download application software (i.e., an “app”) locally to the mobile device. Thus, each PWA described herein may be embodied as a web application or web page that is executable via the MCD 18. Each PWA may appear to the user of the MCD 18 to look and operate similarly to a native application, but as a web application, the PWA does not need to be independently installed prior to execution. An example embodiment of a process executed by the communication engine module 322 and the PWA module 324 is illustrated in FIG. 5, and such a process will be described in detail hereinafter.

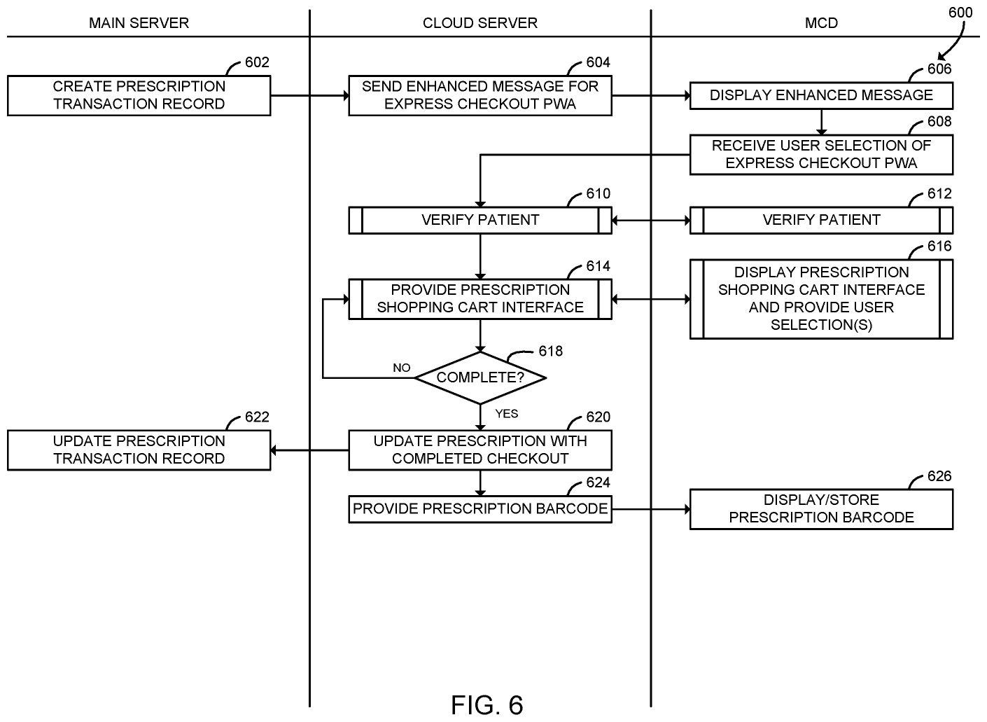

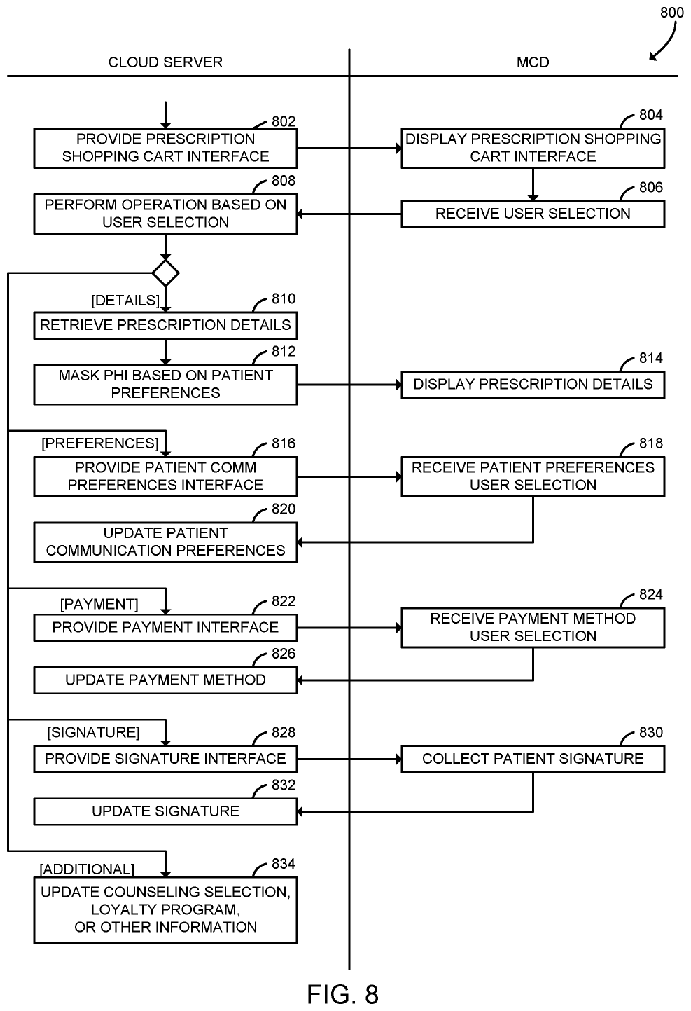

The PWA module 324 further illustratively includes an express checkout module 326, a date of birth verification module 328, a communications preferences module 330, a ready time module 332, a drug recall module 334, a refill reminder module 336, a refill request module 338, a PIN verification module 340, an immunization module 342, and a two-way interaction module 344. The express checkout module 326 is illustratively operable to manage an express checkout process for one or more prescription transactions. An example embodiment of a process executed by the express checkout module 326 is illustrated in FIGS. 6 and 8, and such a process will be described in detail hereinafter.

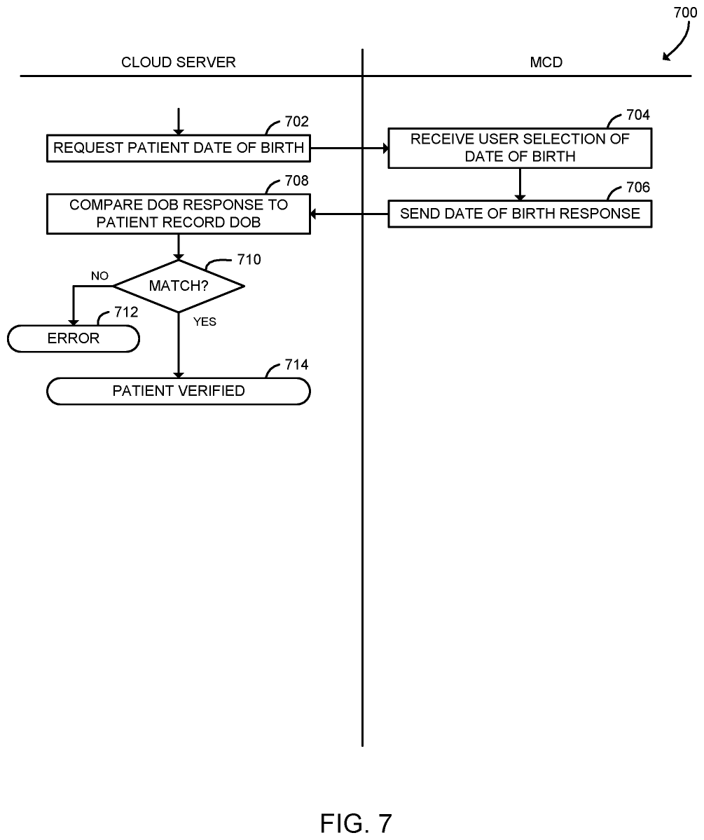

The DOB verification module 328 is illustratively operable to verify the pharmacy patient based on birth date during the express checkout process illustrated in FIG. 6. An example embodiment of a process executed by the DOB verification module 328 is illustrated in FIG. 7, and such a process will be described in detail hereinafter.

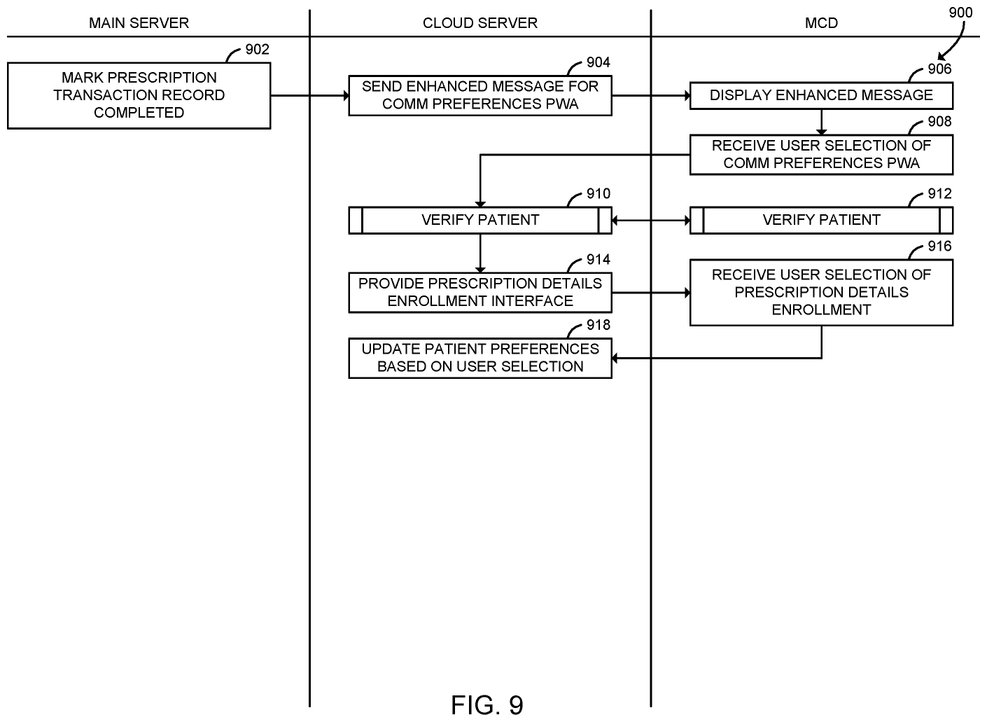

The communications preferences module 330 is illustratively operable to enroll a patient in an enhanced messaging service. An example embodiment of a process executed by the communications preferences module 330 is illustrated in FIG. 9, and such a process will be described in detail hereinafter.

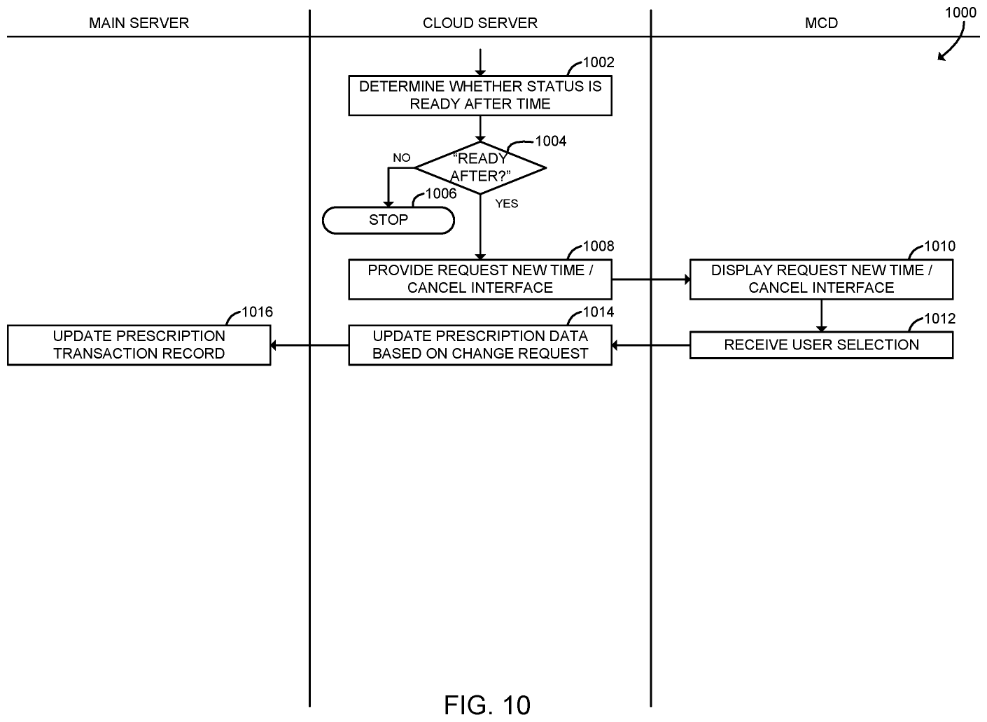

The ready time module 332 is illustratively operable to manage ready times or otherwise update prescriptions. An example embodiment of a process executed by the ready time module 332 is illustrated in FIG. 10, and such a process will be described in detail hereinafter.

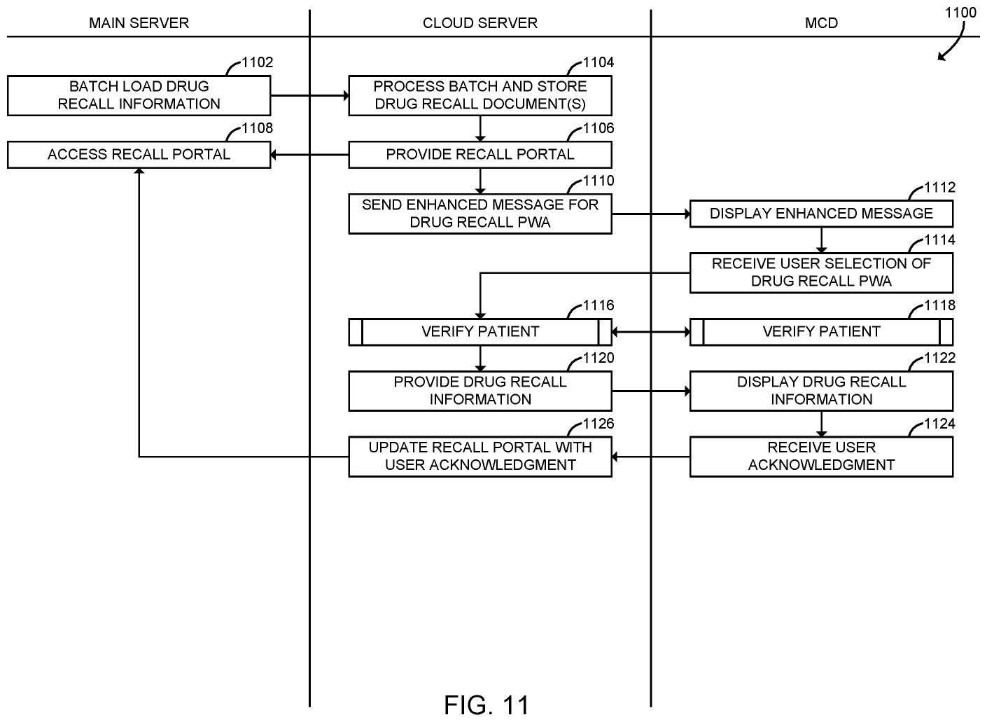

The drug recall module 334 is illustratively operable to manage a drug recall notification process. The drug recall portal 346 is operable to provide drug recall notification information to the main server 14. An example embodiment of a process executed by the drug recall module 334 is illustrated in FIG. 11, and such a process will be described in detail hereinafter.

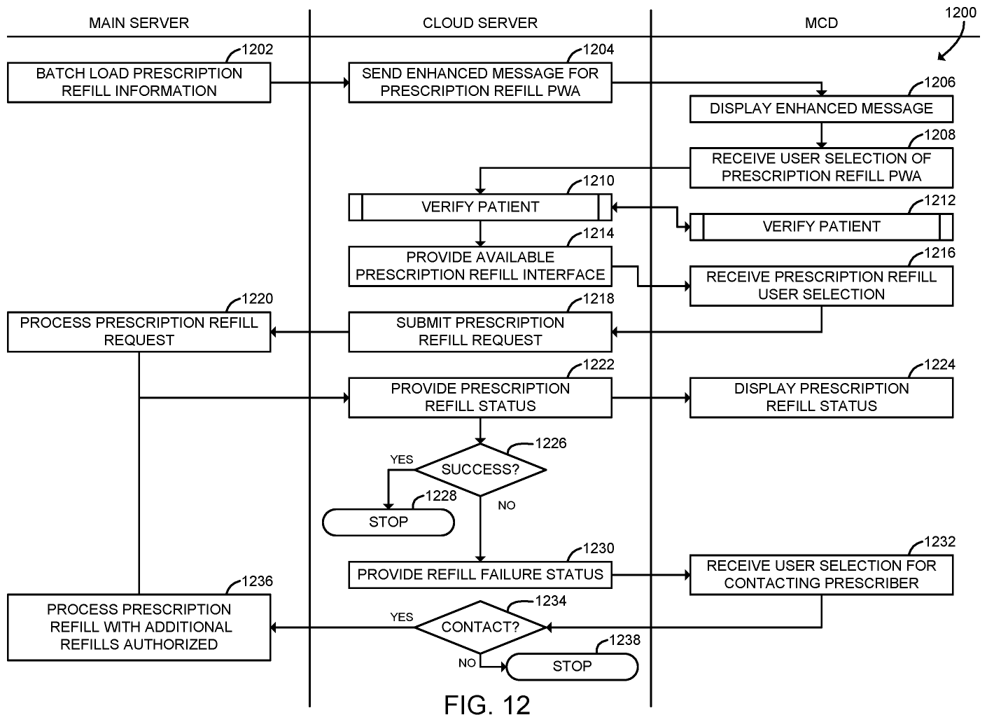

The refill reminder module 336 is illustratively operable to perform a refill reminder process. An example embodiment of a process executed by the refill reminder module 336 is illustrated in FIG. 12, and such a process will be described in detail hereinafter.

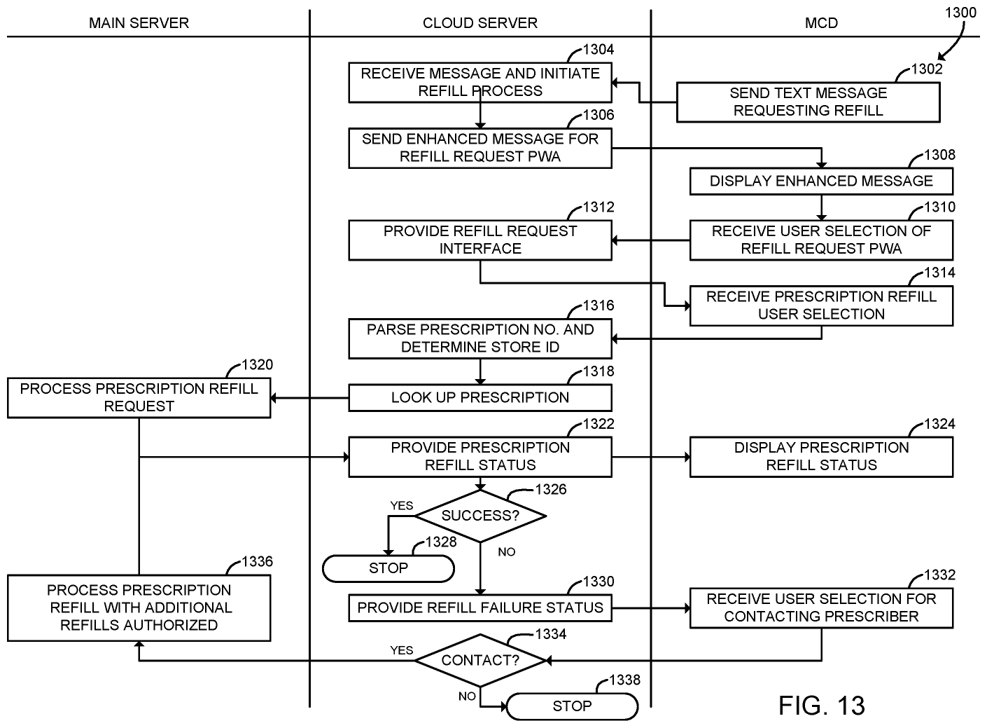

The refill request module 338 is illustratively operable to perform a prescription refill process. An example embodiment of a process executed by the refill request module 338 is illustrated in FIG. 13, and such a process will be described in detail hereinafter.

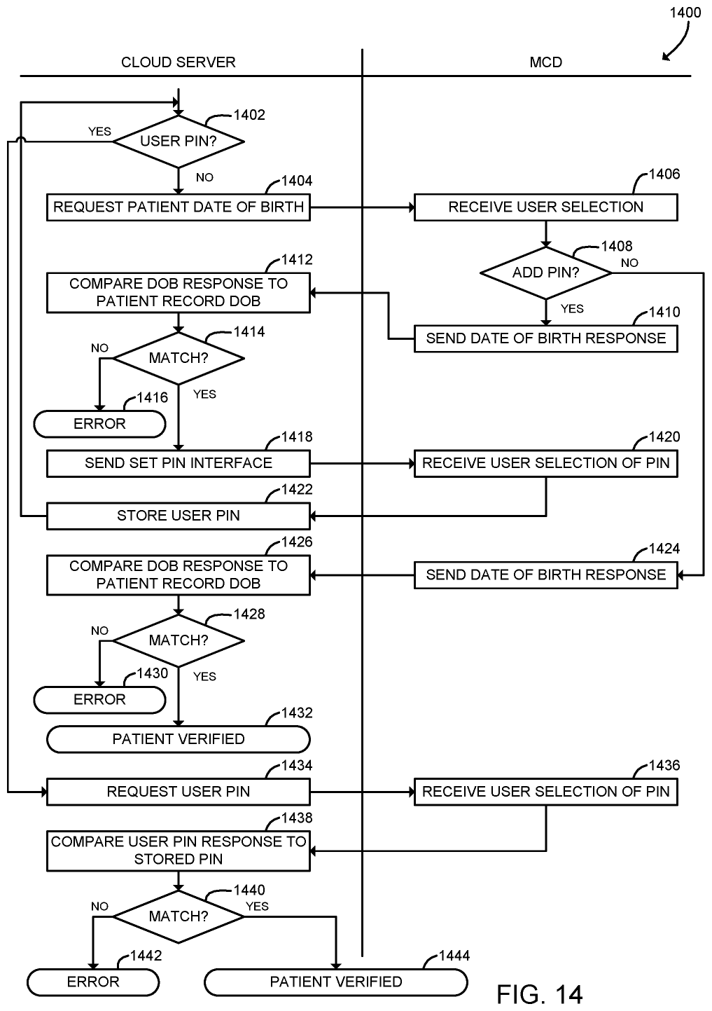

The PIN verification module 340 is illustratively operable to verify the pharmacy patient based on birth date or a user-provided PIN during the express checkout process illustrated in FIG. 6 or during another process. An example embodiment of a process executed by the PIN verification module 340 is illustrated in FIG. 14, and such a process will be described in detail hereinafter.

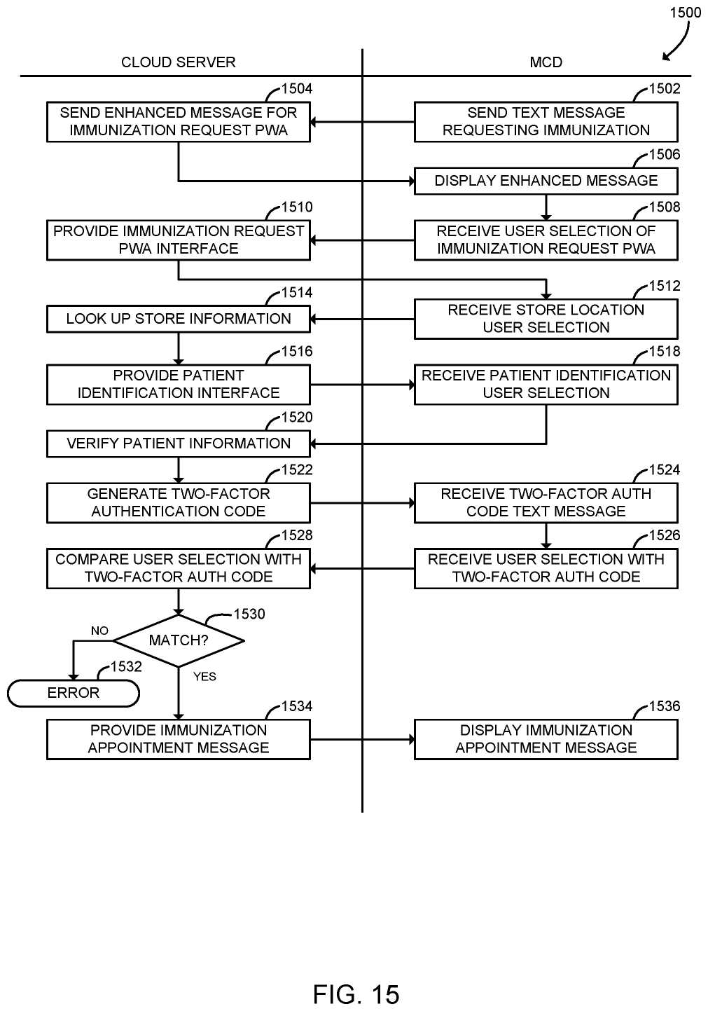

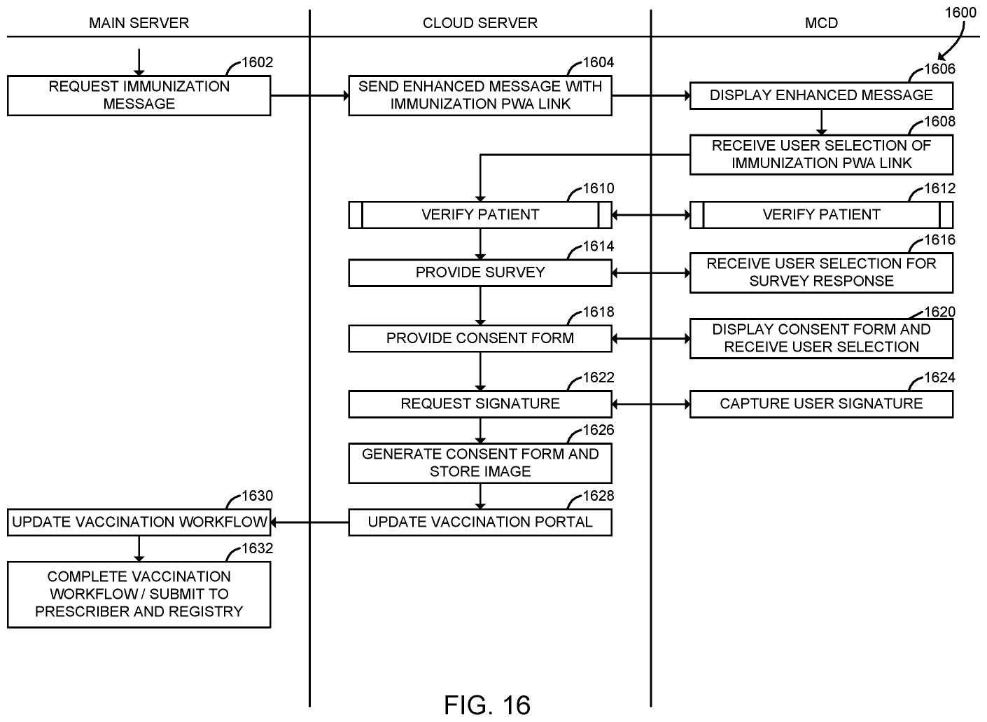

The immunization module 342 is illustratively operable to perform an immunization request process and an immunization preparation process. Example embodiments of processes executed by the immunization module 342 are illustrated in FIGS. 15 and 16, and such processes will be described in detail hereinafter.

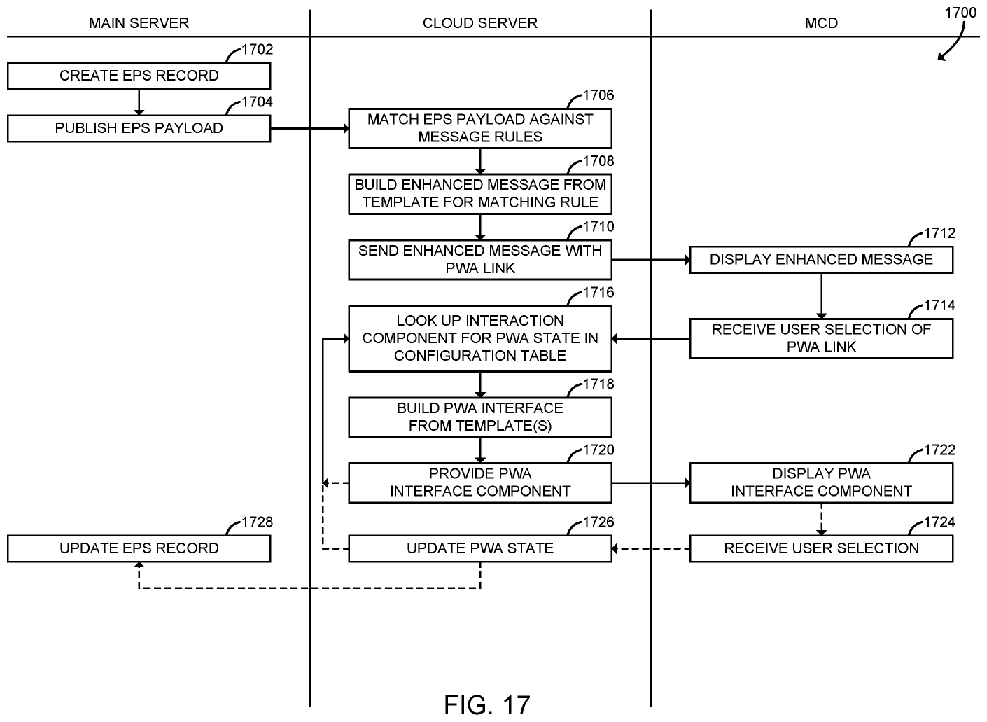

The two-way interaction module 344 is illustratively operable to perform a dynamic two-way communication process during one or more other processes such as the immunization preparation process of FIG. 16. An example embodiment of a process executed by the two-way interaction module 344 is illustrated in FIG. 17, and such a process will be described in detail hereinafter.

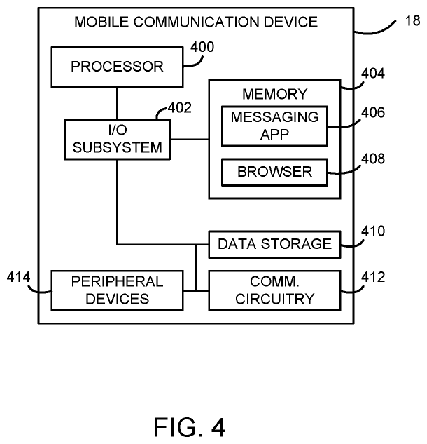

Referring now to FIG. 4, an embodiment of one of the mobile communication devices 18 illustrated in FIG. 1 is shown, which includes components similar to the main server 14 and also to the cloud server 58, such as a processor 400, an I/O subsystem 402, a memory 404 including a messaging application 406 and a web browser 408, a data storage device 410, communication circuitry 412 and a number of peripheral devices 414. In some embodiments, each of the foregoing components may be identical to corresponding components of the local hub server 32₁ and/or main server 14 described above, and a detailed explanation of such components will not be repeated here for brevity. In other embodiments, any of the one or more mobile communication devices 18₁-18_{J} may be configured differently than the local hub server 32₁ described above. It will be appreciated that the mobile communication device 18 may include other components, sub-components, and devices commonly found in a smart phone and/or computing device.

The memory 404 illustratively includes the messaging application 406 in the form of, e.g., instructions executable by the processor 400 to perform conventional text messaging (e.g., SMS) communications over a mobile network. The memory 404 further illustratively includes the web browser 408 in the form of, e.g., instructions executable by the processor 400 to perform conventional web browsing and rendering operations and may be embodied as a native web browser, embedded web browser, browser engine, web application engine, or other web renderer of the mobile communications device 18. In particular, the web browser 408 may execute one or more PWAs received from the cloud server 58. As described above, each PWA may be embodied as a web application including one or more web pages, scripts, or other web resources that are executable or otherwise accessible by the MCD 18. Each PWA may appear to the user of the MCD 18 to look and operate similarly to a native application. However, differently from many native applications, as a web application, the PWA does not need to be independently installed by a user prior to execution. Additionally, the web browser 408 may conventionally allow the PWA to receive user selections such as text entry, touch screen input, voice input, camera input, or other user input interactions, from the one or more peripheral devices 414. Such user selections may be communicated by the web browser 408 to the cloud server 58 as described further below.

The communication circuitry 412 illustratively includes conventional wireless communication circuitry 412. In some embodiments, the wireless communication circuitry 412 is configured to conduct and facilitate cellular telephone communications with other cellular and land-based communication devices. In some embodiments, the wireless communication circuitry 412 is configured to conduct and facilitate communication with the cloud server 58 via the network 16. In some embodiments, the wireless communication circuitry 412 is configured to access the network 16 via at least one hotspot established in any of the brick-and-mortar stores 22₁-22_{L} by a corresponding at least one WiFi Access Point 28. The wireless communication circuitry 412 may illustratively include conventional communication circuitry for conducting and facilitating any such communication, and examples of such conventional communication circuitry include, but are not limited to, one or more conventional radio frequency (RF) transceivers configured to receive and transmit signals at multiple radio frequencies, one or more conventional modem or other communication circuits configured to access and conduct communications via the Internet, and the like. The mobile communication device 18 may illustratively use any suitable communication protocol via the network 16 or other network to communicate with the cloud server 58 and/or with other cellular and land-based communication devices.

Referring now to FIG. 5, a simplified flow diagram is shown depicting an embodiment of a process 500 for enhanced messaging incorporating progressive web applications (PWAs). As indicated by the framework of the process 500 illustrated in FIG. 5, a portion of the process 500, i.e., the portion to the left of the left-most vertical line and centered under the heading “Main Server,” illustratively represents one or more software applications executed by the processor 46 of the main server 14. In one embodiment, this portion of the process 500 is or includes one or more of the modules stored in the EPS access module 206 (see FIG. 2) in the form of instructions executable by the processor 46 of the main server 14. The process steps of this portion of the process 500 will be described below for purposes of this disclosure as being executed by the processor 46 of the main server 14. In some alternate embodiments, e.g., that may or may not include a main server 14, this portion of the process 500 may alternatively be stored in the memory 38 (and/or data storage 40) of one or more of the local servers 32₁-32_{L} in the form of instructions executable by the processor 34 of the one or more local servers 32₁-32_{L}, stored in the memory of one of the point-of-sale systems 26₁-26_{M} within one or more of the brick-and-mortar enterprise stores or outlets in the form of instructions executable by a processor associated with any such point-of-sale system 26₁-26_{M} and/or stored in a memory and executable by a processor of another system external to or supplemental to the system 10 illustrated FIG. 1.

Another portion of the process 500, i.e., the portion between the left-most vertical line and the right-most vertical line in FIG. 5, and centered under the heading “Cloud Server,” illustratively represents one or more software applications executed by a processor 60 of the cloud server 58. In one embodiment, this portion of the process 500 is or includes the communication engine module 322 and the PWA module 324 stored in the memory 64 (and/or data storage 66) of the cloud server 58 in the form of instructions executable by the processor 60 of the cloud server 58 (see FIG. 3). The process steps of this portion of the process 500 will be described below for purposes of this disclosure as being executed by the processor 60 of the cloud server 58.

Still another portion of the process 500, i.e., the portion to the right of the right-most vertical line in FIG. 5, and centered under the heading “MCD,” illustratively represents one or more software applications executed by the processor of a patient's mobile communication device 18, i.e., one of the mobile communication devices 18₁-18j associated with a patient of the retail pharmacy enterprise 12. In one embodiment, this portion of the process 500 is or includes the messaging application 406 and the web browser 408 stored in the memory 404 (and/or the data storage 410) of the patient's mobile communication device 18 (see FIG. 4) in the form of instructions executable by the processor 400 of the patient's mobile communication device 18. The process steps of this portion of the process 500 will be described below for purposes of this disclosure as being executed by the processor 400 of the patient's mobile communication device 18.

It will further be understood that portions of the process 500 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 500 illustrated in FIG. 5 starts in step 502, in which the main server 14 (e.g., the processor 46 of the main server 14) is operable to create or update an enterprise pharmacy system (EPS) 202 record, such as one or more items of prescription transaction data 204. The EPS 202 may include a database, portal, and/or other systems used by the retail pharmacy enterprise 12 to manage workflow, track orders, manage patient relations, and perform other tasks. In the illustrative embodiment the main server 14 may create or update an EPS 202 record in response to a patient requesting to fill one or more prescriptions, for example at a retail location 22 or otherwise.

In step 504, the main server 14 publishes an EPS 202 payload that includes data representing the created and/or updated EPS 202 record. The main server 14 may publish the EPS 202 payload periodically, responsively, or otherwise, and may use any appropriate technique to publish the EPS 202 payload. As shown, the published EPS 202 payload is received and processed by the cloud server 58. In the illustrative embodiment, the main server 14 may publish the EPS 202 payload using an EPS Outbound Patient Notifications (EOPN) service as one or more SOAP (e.g., Simple Object Access Protocol) messages to the cloud server 58. The cloud server 58 may store the EPS 202 payload and/or data extracted from the EPS 202 payload into one or more data stores 302 or other data collections maintained by or accessible to the cloud server 58.

In step 506, the cloud server 58 (e.g., the processor 60 of the cloud server 58) matches the EPS 202 payload against multiple communication rules. Each of the communication rules may be embodied as any conditions, database queries, or other computer code that may be evaluated against the EPS 202 payload (or one or more of the other data stores 302) to determine whether that data matches the communication rule. In some embodiments, each communication rule and/or a rules engine may be embodied as a function, lambda, or other code executed by the cloud server 58 using a function as a service (FaaS) platform or other “serverless” architecture.

In step 508, the cloud server 58 builds an enhanced message from one or more templates of message types associated with a communication rule that matches the EPS 202 payload. Each template may be associated with a different patient interaction or part of a different patient interaction. Each template may include static text or other content used to communicate with the patient, and in some embodiments may incorporate data from the EPS 202 payload and/or other data stores 302 maintained by the cloud server 58 and/or the main server 14. Illustratively, the cloud server 58 maintains templates for certain pre-configured messages or other interactions. One potential embodiment of message types and associated identification numbers is shown below in Table 1.

TABLE 1
Message types for enhanced messaging.

| | |
|---|---|
| 1 | (Reserved) |
| 2 | Issue |
| 3 | OOS |
| 4 | Partial Ready |
| 5 | Ready |
| 6 | SOLD |
| 7 | RTS |
| 8 | Issue Specialty |
| 9 | Process |
| 10 | Custom Text |
| 11 | Delayed Delivery |
| 12 | Amount Due Update Message |
| 13 | OOS Date Update Message Sent |
| 14 | Promise Time Update Message |
| 15 | EnhancedSMS |
| 16 | Manual Express resend |
| 17 | MedSync |
| 18 | RECALL |
| 19 | Refill Reminder |
| 20 | Cancelled by prescriber |
| 21 | Ready - Schedule 2-5 |
| 22 | Aging 1 |
| 23 | Aging 2 |
| 24 | Aging 3 |
| 25 | Aging 4 |
| 26 | Aging 5 |
| 27 | Sync 1 |
| 28 | Sync 2 |
| 29 | Sync 3 |
| 30 | Sync 4 |
| 31 | Specialty Program |
| 32 | Retail Program |
| 33 | Immunization Program |
| 34 | Immunization Add On |
| 35 | Retail Program |
| 36 | Immunization In-Process |
| 37 | Immunization Ready |
| 38 | Immunization Sold |
| 39 | Immunization Cancel |
| 40 | Service Greeting Text |
| 41 | Two Factor Code |
| 42 | Two Factor Code NEW PATIENT |
| 43 | Clinic Invite |

In step 510, the cloud server 58 sends the enhanced message to the patient. The cloud server 58 sends the message via short message service (SMS) or other messaging service to a phone number associated with the EPS 202 payload (and, thus, the patient). For example, the cloud server 58 may send the message to a phone number provided by the patient when initially requesting the prescription fill. The enhanced message includes an application address, e.g., in the form of a link or other selectable application address, to a progressive web application (PWA). The link may be embodied as a uniform resource indicator (URI), uniform resource locator (URL), or other address that identifies a particular PWA or other web application resource provided by the cloud server 58. In some embodiments, the link to the PWA may include a unique identifier associated with the particular patient, such as a random identification number generated by the cloud server 58.

In step 512, the MCD 18 associated with the patient receives and displays the enhanced message including the PWA link. The enhanced message may be displayed, for example, using the native messaging application 406 provided by the MCD 18. Thus, the enhanced message may be displayed and/or otherwise accessed without requiring the patient to download or install a custom application on the MCD 18. In step 514, the MCD 18 receives a patient selection of the PWA link, i.e., the patient manually selects the PWA displayed on the display of the MCD 18 via conventional patient interaction with the MCD 18. For example, the patient may tap, click, or otherwise select the PWA link displayed in the enhanced message.

In response to the patient selection, in step 516 the MCD 18 executes the selected PWA. For example, the MCD 18 may load the selected PWA link in the native web browser 408 provided by the MCD 18. Thus, the MCD 18 may execute the selected PWA without requiring the user to download or install a custom application on the MCD 18. In step 518, and in coordination with step 516, the cloud server 58 also executes the selected PWA. The cloud server 58 may, for example, provide one or more web pages, scripts, or other interface resources to the web browser 408 of the MCD 18. The cloud server 58 may receive user selections, form submissions, or other data uploaded by the MCD 18. As part of executing the application, the cloud server 58 may access one or more of the data stores 302 maintained by the cloud server 58 and perform other application tasks. In some embodiments, the cloud server 58 may also provide updated data or issue other requests to the main server 14. In those embodiments, in step 520 the main server 14 may update the EPS 202 record based on data provided by the cloud server 58. In any case, the could server 58, with the MCD 18, execute the selected PWA, i.e., the PWA associated with the application address sent by the could server 58 to the MCD 18, via which the retail pharmacy enterprise 12 interactively, i.e., between the cloud server 58 and the patient via the patient's MCD 18, provides one or more retail pharmacy services to the patient, some non-limiting examples of which are described in detail below. After executing the PWA, the process 500 is completed. The process 500 may be repeated, for example, in response to further updates in one or more EPS 202 records.

### Express Checkout Process

Referring now to FIG. 6, a simplified flow diagram is shown depicting an embodiment of a process 600 for express checkout using enhanced messaging with an express checkout PWA. As described further below, the process 600 may incorporate and/or extend functionality provided by the enhanced messaging process 500 described above. As indicated by the framework of the process 600 illustrated in FIG. 6, a portion of the process 600, i.e., the portion to the left of the left-most vertical line and centered under the heading “Main Server,” illustratively represents one or more software applications executed by the processor 46 of the main server 14. In one embodiment, this portion of the process 600 is or includes one or more of the modules stored in the EPS access module 206 (see FIG. 2) in the form of instructions executable by the processor 46 of the main server 14. The process steps of this portion of the process 600 will be described below for purposes of this disclosure as being executed by the processor 46 of the main server 14. In some alternate embodiments, e.g., that may or may not include a main server 14, this portion of the process 600 may alternatively be stored in the memory 38 (and/or data storage 40) of one or more of the local servers 32₁-32_{L} in the form of instructions executable by the processor 34 of the one or more local servers 32₁-32_{L}, stored in the memory of one of the point-of-sale systems 26₁-26_{M} within one or more of the brick-and-mortar enterprise stores or outlets in the form of instructions executable by a processor associated with any such point-of-sale system 26₁-26_{M} and/or stored in a memory and executable by a processor of another system external to or supplemental to the system 10 illustrated FIG. 1.

Another portion of the process 600, i.e., the portion between the left-most vertical line and the right-most vertical line in FIG. 6, and centered under the heading “Cloud Server,” illustratively represents one or more software applications executed by a processor 60 of the cloud server 58. In one embodiment, this portion of the process 600 is or includes the communication engine module 322 and the express checkout module 326 stored in the memory 64 (and/or data storage 66) of the cloud server 58 in the form of instructions executable by the processor 60 of the cloud server 58 (see FIG. 3). The process steps of this portion of the process 600 will be described below for purposes of this disclosure as being executed by the processor 60 of the cloud server 58.

Still another portion of the process 600, i.e., the portion to the right of the right-most vertical line in FIG. 6, and centered under the heading “MCD,” illustratively represents one or more software applications executed by the processor of a patient's mobile communication device 18, i.e., one of the mobile communication devices 18₁-18j associated with a patient of the retail pharmacy enterprise 12. In one embodiment, this portion of the process 600 is or includes the messaging application 406 and the web browser 408 stored in the memory 404 (and/or the data storage 410) of the patient's mobile communication device 18 (see FIG. 4) in the form of instructions executable by the processor 400 of the patient's mobile communication device 18. in the form of instructions executable by the processor of the patient's mobile communication device 18. The process steps of this portion of the process 600 will be described below for purposes of this disclosure as being executed by the processor 400 of the patient's mobile communication device 18.

It will further be understood that portions of the process 600 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 600 illustrated in FIG. 6 starts in step 602, in which the main server 14 (e.g., the processor 46 of the main server 14) is operable to create a prescription transaction record. The prescription transaction record may be embodied as any database record or other data describing a prescription transaction originated by a patient, prescriber, or other entity. Thus, the prescription transaction record may include data indicative of a patient as well as one or more prescriptions associated with the patient. The prescription transaction may be embodied as one or more records or other data maintained by an enterprise pharmacy system (EPS) 202 maintained by the main server 14. As described above in connection with FIG. 5, the main server 14 may publish a data payload indicative of the prescription transaction record to the cloud server 58. The data payload may be received by the cloud server 58 and stored in the express cart collection 304 or other data store 302 maintained by the cloud server 58.

In step 604, the cloud server 58 (e.g., the processor 60 of the cloud server 58) sends an enhanced message including a link to an express checkout progressive web application (PWA) to the MCD 18 associated with the patient. As described above in connection with FIG. 5, the enhanced message may be sent via SMS or other messaging service to a phone number associated with the prescription transaction record. For example, the phone number may be supplied by the patient when initially requesting that the prescription be filled. Also as indicated above, the enhanced message includes a link such as a URI or URL that identifies the express checkout PWA provided by the cloud server 58. In some embodiments, the PWA link may also include a unique identifier generated by the cloud server 58 and associated with the particular prescription transaction. As described further below, this unique identifier may be used by the cloud server 58 to associate PWA interactions with the MCD 18 with a particular prescription transaction, for example by referencing one or more records stored in the express cart collection 304.

In step 606, the MCD 18 associated with the patient receives and displays the enhanced message including the PWA link. The enhanced message may be displayed, for example, using the native messaging application 406 provided by the MCD 18. Thus, the enhanced message may be displayed and/or otherwise accessed without requiring the patient to download or install a custom application on the MCD 18. In step 608, the MCD 18 receives a user selection of the express checkout PWA link. For example, the patient may tap, click, or otherwise select the PWA link displayed in the enhanced message. The MCD 18 may send a request for the URI or other resource identified by the PWA link to the cloud server 58.

In steps 610 and 612, the cloud server 58 and the MCD 18 cooperate to perform a verification process to verify the identity of the patient. For example, the cloud server 58 may send a challenge request to the MCD 18 that requests a date of birth or other verifying information associated with the patient. In response, the MCD 18 may send a response including a user selection of the date of birth or other verifying information. It should be understood that the date of birth or other verifying information may have been previously provided by the patient, for example when initially requesting the prescription fill. Thus, the date of birth or other verifying information may be known by the main server 14 and/or the cloud server 58 prior to executing the verification process. If the patient is not successfully verified, the process 600 may be repeated or halted. In some embodiments, the cloud server 58 may lock the prescription transaction or otherwise prevent execution of the express checkout process if verification fails after more than a predetermined number of attempts. If the patient is successfully verified, the process 600 advances to step 614. At least one potential embodiment of a verification process is described further below in connection with FIG. 7.

In step 614, the cloud server 58 provides a prescription shopping cart interface to the MCD 18. The shopping cart interface may provide information regarding the prescription transaction, including drug information, prices, ready time, and other status information. That prescription transaction information may be stored in the express cart collection 304 or other data storage maintained by the cloud server 58. In some embodiments, when providing the shopping cart interface, the cloud server 58 may mask protected health information (PHI) according to one or more user communications preferences associated with the patient. Those communications preferences may be stored in the patient preference table 306, described further below. The shopping cart interface may also request signatures or other information from the patient, and the cloud server 58 may store or otherwise process such information, for example in blob storage 320. In step 616, the MCD 18 displays the prescription shopping cart interface and may receive one or more user selections from the patient. The MCD 18 may provide any such user selections to the cloud server 58. One potential embodiment of a process for providing and displaying the prescription shopping cart interface is described further below in connection with FIG. 8.

In step 618, the cloud server 58 determines whether the shopping cart checkout process has been completed. The cloud server 58 may determine, for example, whether all required payment information, survey information, or other required information has been received from the MCD 18, and whether the patient has requested to complete checkout. If not, the process 600 loops back to step 614 to continue providing the shopping cart interface. If the checkout process is completed, the process 600 advances to step 620.

In step 620, the cloud server 58 updates the prescription transaction with completed checkout information. For example, the cloud server 58 may replace a record in the express cart collection 304 with a duplicate payload that includes a flag indicating that checkout is complete. The cloud server 58 may also provide updated checkout information to the main server 14. In step 622, the main server 14 updates the associated prescription transaction record in the EPS 202 with completed checkout information. The main server 14 and/or an associated local hub server 32 may provide the completed checkout information to the appropriate retail outlet 22.

In step 624, the cloud server 58 provides a prescription barcode to the MCD 18. The barcode may identify the completed prescription transaction, for example by representing an associated identification number or other unique identifier. The cloud server 58 may provide the bar code as part of a PWA, a web page, or other interface transmitted to the MCD 18. In step 626, the MCD 18 displays and/or stores the prescription barcode for use by the patient. For example, the MCD 18 may display the PWA received from the cloud server 58 using a native web browser of the MCD 18. In some embodiments, the MCD 18 may store the prescription barcode to a native “wallet” application or other application used to manage barcodes. In use, the user of the MCD 18 may present the prescription barcode at the appropriate retail outlet 22 in order to pick up the completed prescription.

### Date of Birth Verification Process

Referring now to FIG. 7, a simplified flow diagram is shown of an embodiment 700 of the date of birth verification process executed by the cloud server 58 and MCD 18 at steps 610, 612 of the process 600. As indicated by the framework of the process 700 illustrated in FIG. 7, a portion of the process 700, i.e., the portion to the left of the vertical line and centered under the heading “Cloud Server,” illustratively represents one or more software applications executed by a processor 60 of the cloud server 58. In one embodiment, this portion of the process 700 is or includes the DOB verification module 328 stored in the memory 64 (and/or data storage 66) of the cloud server 58 in the form of instructions executable by the processor 60 of the cloud server 58 (see FIG. 3). The process steps of this portion of the process 700 will be described below for purposes of this disclosure as being executed by the processor 60 of the cloud server 58.

Still another portion of the process 700, i.e., the portion to the right of the vertical line in FIG. 7, and centered under the heading “MCD,” illustratively represents one or more software applications executed by the processor of a patient's mobile communication device 18, i.e., one of the mobile communication devices 18₁-18_{J} associated with a patient of the retail pharmacy enterprise 12. In one embodiment, this portion of the process 700 is or includes is or includes the web browser 408 stored in the memory 404 (and/or the data storage 410) of the patient's mobile communication device 18 (see FIG. 4) in the form of instructions executable by the processor 400 of the patient's mobile communication device 18. The process steps of this portion of the process 700 will be described below for purposes of this disclosure as being executed by the processor 400 of the patient's mobile communication device 18.

It will further be understood that portions of the process 700 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 700 illustrated in FIG. 7 starts in step 702, in which the cloud server 58 (e.g., the processor 60 of the cloud server 58) is operable to request the date of birth from the patient. As discussed above, to request the patient's date of birth, the cloud server 58 may transmit a progressive web application (PWA), web page, or other challenge interface to the MCD 18 associated with the patient.

In step 704, the MCD 18 receives a user selection of the patient's birth date. To receive the user selection, the MCD 18 may display the PWA, web page, or other challenge interface received from the cloud server 58. The challenge interface may include a date picker, text box, or other user interface elements configured to receive a date of birth from the user. The MCD 18 may display the interface using the native web browser 408. In step 706, the MCD 18 sends a response to the cloud server 58 that includes the date of birth user selection received from the user.

In step 708, the cloud server 58 compares the date of birth response received from the MCD 18 to a date of birth previously associated with the patient. For example, as described above, the patient may supply the date of birth when initially requesting the prescription fill or otherwise initiating the prescription transaction. The date of birth may be stored by the cloud server 58 in the express cart collection 304 and/or one or more other data stores 302 maintained by the cloud server 58. Additionally or alternatively, in some embodiments, the cloud server 58 may retrieve the date of birth from the main server 14 (e.g., from the EPS 202).

In step 710, the cloud server 58 determines whether the birth date supplied by the MCD 18 matches the previously stored birth date. If not, the process 700 branches to step 712, in which the cloud server 58 indicates a verification error. In some embodiments, the process 700 may be repeated one or more times in order to allow the user of the MCD 18 to attempt to repeat verification. After exceeding a predetermined number of attempts, (e.g., three attempts), the process 700 may be locked for a predetermined time period (e.g., two hours), preventing further verification attempts.

Referring back to step 710, if the birth date supplied by the MCD 18 matches the previously stored birth date, the process 700 advances to step 714, in which the patient is successfully verified. After successful verification, the system 10 may proceed with the current PWA process. For example, as described above, after successful verification, the process 600 may continue executing in step 614.

### Shopping Cart Process

Referring now to FIG. 8, a simplified flow diagram is shown of an embodiment 800 of the shopping cart process executed by the cloud server 58 and the MCD 18 at steps 614, 616 of the process 600. As indicated by the framework of the process 800 illustrated in FIG. 8, a portion of the process 800, i.e., the portion to the left of the vertical line and centered under the heading “Cloud Server,” illustratively represents one or more software applications executed by a processor 60 of the cloud server 58. In one embodiment, this portion of the process 800 is or includes the express checkout module 326 stored in the memory 64 (and/or data storage 66) of the cloud server 58 in the form of instructions executable by the processor 60 of the cloud server 58 (see FIG. 3). The process steps of this portion of the process 800 will be described below for purposes of this disclosure as being executed by the processor 60 of the cloud server 58.

Still another portion of the process 800, i.e., the portion to the right of the vertical line in FIG. 8, and centered under the heading “MCD,” illustratively represents one or more software applications executed by the processor of a patient's mobile communication device 18, i.e., one of the mobile communication devices 18₁-18_{J} associated with a patient of the retail pharmacy enterprise 12. In one embodiment, this portion of the process 800 is or includes the web browser 408 stored in the memory 404 (and/or the data storage 410) of the patient's mobile communication device 18 (see FIG. 4) in the form of instructions executable by the processor 400 of the patient's mobile communication device 18. The process steps of this portion of the process 800 will be described below for purposes of this disclosure as being executed by the processor 400 of the patient's mobile communication device 18.

It will further be understood that portions of the process 800 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 800 illustrated in FIG. 8 starts in step 802, in which the cloud server 58 (e.g., the processor 60 of the cloud server 58) is operable to provide a shopping cart user interface to the MCD 18. To provide the shopping cart interface, the cloud server 58 may look up one or more data payloads or other data records relating to the prescription transaction. Those data records may include prescription details (e.g., drug name, prescription number, promised completion time), payment method details, status of required information (e.g., patient signature, counseling information), or other information relating to the prescription transaction (e.g., loyalty card number or other data). The cloud server 58 may, for example, retrieve one or more records from the express cart collection 304 or other data storage maintained by the cloud server 58. The cloud server 58 may provide the shopping cart interface as a PWA, a web page, or other user interface transmitted to the MCD 18. In step 804, the MCD 18 displays the prescription shopping cart interface, for example using the native web browser 408 of the MCD 18.

In step 806, the MCD 18 receives a user selection from the user of the MCD 18. The user selection may include input of text or other data, selection of a hyperlink, or other user interface action initiated by the user. The MCD 18 responds with the user selection to the cloud server 58.

In step 808, the cloud server 58 performs one or more operations based on the user selection received from the MCD 18. The cloud server 58 may select among multiple operations based on, for example, a URI or other resource identifier received from the MCD 18 or based upon data received from the MCD 18. If the cloud server 58 determines that the user selected additional prescription details, for example in response to selection of an information button or other user interface element, the process 800 advances to block 810.

In step 810, the cloud server 58 retrieves additional prescription details for the requested prescription(s). The cloud server 58 may retrieve, for example, prescriber information (e.g., prescriber name, address, phone number, etc.), transaction data (e.g., prescription number, filled date, etc.), prescription data (e.g., drug name, days supply, quantity dispensed, drug unit, written date, last filled date, refills remaining, expiration date, etc.), pharmacy data (e.g., promised fill time, product ID), or other data. The prescription details may be retrieved from the express cart collection 304. In step 812, the cloud server 58 masks protected health information (PHI) based on patient communication preferences. For example, the cloud server 58 may maintain the patient preference table 306 indicating whether a patient has opted in to view prescription details. If the patient has not opted in to viewing prescription details (e.g., the user has not yet responded or has affirmatively declined viewing details), the cloud server 58 may hide or obfuscate prescription details such as drug name, prescription number, written date, last filled date, or other information that could include PHI. After retrieving and potentially masking the prescription details information, the cloud server 58 sends the prescription details to the MCD 18 as a PWA or web page. In step 814, the MCD 18 displays the prescription details received from the cloud server 58. The user may continue to interact with the shopping cart interface as described above.

Referring back to step 808, if the cloud server 58 determines that the user selected to update communication preferences, the process 800 advances to step 816, in which the cloud server 58 provides a patient communication preferences interface to the MCD 18. The patient communication preferences interface may expose various options included in the patient preference table such as various privacy or personalization options, including opting in to viewing prescription details. Additionally or alternatively, in some embodiments, the opt-in interface may be exposed through a separate enhanced messaging interaction described below in connection with FIG. 9. The cloud server 58 sends the patient communications preferences interface to the MCD 18 as a PWA or a web page. In step 818, the MCD 18 displays the patient communications preferences interface and receives a user selection including one or more patient preferences. The MCD 18 sends a response to the cloud server 58 including the user selection, and in step 820 the cloud server 58 updates the patient communications preferences. For example, the cloud server 58 may store the preferences received from the MCD 18 into the patient preferences table 306. After updating patient preferences, the user may continue to interact with the shopping cart interface as described above.

Referring back to step 808, if the cloud server 58 determines that the user has selected to update a payment method, the process 800 advances to step 822, in which the cloud server 58 provides a payment interface to the MCD 18. As described above, the cloud server 58 may send the payment interface to the MCD as a PWA or a web page. In step 824, the MCD 18 displays the payment interface and receives a user selection indicative of a payment method. For example, the user may provide credit card information, debit card information, or other payment method information. The MCD 18 sends a response including the payment method user selection to the cloud server 58, and in step 826 the cloud server 58 updates payment method information. The cloud server 58 may, for example, update credit card information in the express cart collection 304 or other data storage maintained by the cloud server 58. After updating payment information, the user may continue to interact with the shopping cart interface as described above.

Referring back to step 808, if the cloud server 58 determines that the user has selected to provide a signature, the process 800 advances to step 828, in which the cloud server 58 provides a signature interface to the MCD 18. The cloud server 58 may send the signature interface to the MCD 18 as a PWA or a web page. In step 830, the MCD 18 displays the signature interface and collects a patient signature from the user. The signature may be collected, for example, using a touch screen or other interface device of the MCD 18. After collecting the signature, the MCD 18 sends data indicative of the signature (e.g., one or more image files) to the cloud server 58. In step 832, the cloud server 58 updates a patient signature. For example, the cloud server 58 may store the image received from the cloud server 58 in the blob storage 320 as a binary “blob,” in bulk storage, as an image file, or in another storage format. After collecting the signature, the user may continue to interact with the shopping cart interface as described above.

Referring back to step 808, if the cloud server 58 receives additional information from the MCD 18, the process 800 advances to step 834. For example, in some embodiments, the user may indicate whether the patient has any questions for the pharmacist or otherwise requests additional counseling. As another example, the user may indicate membership in an enterprise membership services (EMS) program or other loyalty program.

Customers may elect to participate in an enterprise membership services (EMS) program offered, managed and maintained by the retail pharmacy enterprise 12, by establishing a user account (which may be referred to herein as an “EMS account” or “customer account”) within the server 14, which user account may in some cases be an individual account accessible only by an individual person, e.g., an individual customer, and in other cases may be a group or “household” account accessible by each of a plurality of members of a predefined group of persons, e.g., members of a family or household, one or more employees of a business enterprise, etc. MPERKS®, a virtual customer coupon collection and redemption program offered to customers by Meijer, Inc. of Grand Rapids, Mich., is an example of one such EMS program of the type described herein, although it will be appreciated that any retail enterprise membership service which offers virtual discount coupons and/or other benefits to shopper members, and/or which tracks items purchased by shopper members during item purchase transactions at point-of-sale systems or terminals may be alternatively be used.

The cloud server 58 may receive such additional information as selections from one or more check boxes or other user interface elements provided by the MCD 18. The cloud server 58 may update stored data in the express cart collection 304 or other data table based on the user selection received from the MCD 18. After collecting the additional information, the user may continue to interact with the shopping cart interface as described above.

### Detailed Messaging Enrollment Process

Referring now to FIG. 9, a simplified flow diagram is shown depicting an embodiment of a process 900 for detailed messaging enrollment with a patient preferences PWA. As indicated by the framework of the process 900 illustrated in FIG. 9, a portion of the process 900, i.e., the portion to the left of the left-most vertical line and centered under the heading “Main Server,” illustratively represents one or more software applications executed by the processor 46 of the main server 14. In one embodiment, this portion of the process 900 is or includes one or more of the modules stored in the EPS access module 206 (see FIG. 2) in the form of instructions executable by the processor 46 of the main server 14. The process steps of this portion of the process 900 will be described below for purposes of this disclosure as being executed by the processor 46 of the main server 14. In some alternate embodiments, e.g., that may or may not include a main server 14, this portion of the process 900 may alternatively be stored in the memory 38 (and/or data storage 40) of one or more of the local servers 32₁-32_{L} in the form of instructions executable by the processor 34 of the one or more local servers 32₁-32_{L}, stored in the memory of one of the point-of-sale systems 26₁-26_{M} within one or more of the brick-and-mortar enterprise stores or outlets in the form of instructions executable by a processor associated with any such point-of-sale system 26₁-26_{M} and/or stored in a memory and executable by a processor of another system external to or supplemental to the system 10 illustrated FIG. 1.

Another portion of the process 900, i.e., the portion between the left-most vertical line and the right-most vertical line in FIG. 9, and centered under the heading “Cloud Server,” illustratively represents one or more software applications executed by a processor 60 of the cloud server 58. In one embodiment, this portion of the process 900 is or includes the communication engine module 322 and the communications preferences module 330 stored in the memory 64 (and/or data storage 66) of the cloud server 58 in the form of instructions executable by the processor 60 of the cloud server 58 (see FIG. 3). The process steps of this portion of the process 900 will be described below for purposes of this disclosure as being executed by the processor 60 of the cloud server 58.

Still another portion of the process 900, i.e., the portion to the right of the right-most vertical line in FIG. 9, and centered under the heading “MCD,” illustratively represents one or more software applications executed by the processor of a patient's mobile communication device 18, i.e., one of the mobile communication devices 18₁-18_{J} associated with a patient of the retail pharmacy enterprise 12. In one embodiment, this portion of the process 900 is or includes the messaging application 406 and the web browser 408 stored in the memory 404 (and/or the data storage 410) of the patient's mobile communication device 18 (see FIG. 4) in the form of instructions executable by the processor 400 of the patient's mobile communication device 18. The process steps of this portion of the process 900 will be described below for purposes of this disclosure as being executed by the processor 400 of the patient's mobile communication device 18.

It will further be understood that portions of the process 900 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 900 illustrated in FIG. 9 starts in step 902, in which the main server 14 (e.g., the processor 46 of the main server 14) is operable to mark a prescription transaction record 204 of the EPS 202 as completed. The prescription transaction record may be completed, for example, in response to completion of the express checkout process described above in connection with FIGS. 6-8. As described above, the prescription transaction record may be embodied as any database record or other data describing a prescription fill request originated by a patient, prescriber, or other entity, and in particular may be embodied as one or more records or other data maintained by the EPS 202 maintained by the main server 14. As described above, the main server 14 may publish a data payload indicative of the prescription transaction record to the cloud server 58. The data payload may be received by the cloud server 58 and stored in the enhanced messaging lookup collection 307, data table, or other data storage.

In step 904, the cloud server 58 (e.g., the processor 60 of the cloud server 58) sends an enhanced message including a link to a patient communications preferences progressive web application (PWA) to the MCD 18 associated with the patient. The cloud server 58 may send the enhanced message in response to the data payload matching one or more rules, such as rules determining that the data payload is marked as completed and that an enhanced messaging flag is null (e.g., if the prescription details enrollment process has not previously been completed). As described above, the enhanced message may be sent via SMS or other messaging service to a phone number associated with the prescription transaction record. For example, the phone number may be supplied by the patient when initially requesting that the prescription be filled. Also as indicated above, the enhanced message includes a link such as a URI or URL that identifies the patient preferences PWA provided by the cloud server 58. In some embodiments, the PWA link may also include a unique identifier generated by the cloud server 58 and associated with the particular prescription transaction. As described further below, this unique identifier may be used by the cloud server 58 to associate PWA interactions with the MCD 18 with a particular prescription transaction, for example by referencing one or more records in the enhanced messaging lookup collection 307.

In step 906, the MCD 18 associated with the patient receives and displays the enhanced message including the PWA link. The enhanced message may be displayed, for example, using the native messaging application 406 provided by the MCD 18. Thus, the enhanced message may be displayed and/or otherwise accessed without requiring the patient to download or install a custom application on the MCD 18. In step 908, the MCD 18 receives a user selection of the communications preferences PWA link. For example, the patient may tap, click, or otherwise select the PWA link displayed in the enhanced message. The MCD 18 may send a request for the URI or other resource identified by the PWA link to the cloud server 58.

In steps 910 and 912, the cloud server 58 and the MCD 18 cooperate to perform a verification process to verify the identity of the patient. For example, the cloud server 58 may send a challenge request to the MCD 18 that requests a date of birth or other verifying information associated with the patient. In response, the MCD 18 may send a response including a user selection of the date of birth or other verifying information. To verify the patient, the cloud server 58 and the MCD 18 may perform the verification process 700 illustrated in FIG. 7 and described above.

After verifying the patient, in step 914, the cloud server 58 provides a prescription details enrollment interface to the MCD 18. The patient communication preferences interface may expose various options included in the patient preference table such as various privacy or personalization options, including opting in to viewing prescription details. The enrollment interface may indicate that the patient agrees to transmission of protected health information (PHI), and may include one or more checkboxes or other user interface elements allowing the user to affirmatively agree to enroll in receiving prescription details. The cloud server 58 may provide the prescription details enrollment interface as a PWA, a web page, or other interface.

In step 916, the MCD 18 displays the prescription details enrollment interface and receives one or more user selections regarding enrollment in receiving prescription details. For example, the user may affirmatively select to opt out of receiving prescription details, or the user may affirmatively select to receive prescription details. The MCD 18 sends a response to the cloud server 58 including the user selection.

In step 918, the cloud server 58 updates one or more patient preferences based on the user selection received from the MCD 18. For example, the cloud server 58 may update the patient preferences table 306 with the user selection. The patient preference table 306 may indicate whether the user has affirmatively enrolled in receiving detailed prescription information or whether the user has affirmatively opted out. As described above, the cloud server 58 may mask certain protected health information (PHI) if the user has not affirmatively opted in to displaying prescription details. After updating the patient preferences table 306, the process 900 is completed.

### Update Ready Time Process

Referring now to FIG. 10, a simplified flow diagram is shown depicting an embodiment of a process 1000 for updating ready time for a prescription fill order. The process 1000 may be executed, for example, as part of or in conjunction with the express checkout shopping cart processes illustrated in FIGS. 6, 8 and described above. As such, the process 1000 may be executed after verifying the patient using the date of birth verification process 700 described above in connection with FIG. 7.

As indicated by the framework of the process 1000 illustrated in FIG. 10, a portion of the process 1000, i.e., the portion to the left of the left-most vertical line and centered under the heading “Main Server,” illustratively represents one or more software applications executed by the processor 46 of the main server 14. In one embodiment, this portion of the process 1000 is or includes one or more of the modules stored in the EPS access module 206 (see FIG. 2) in the form of instructions executable by the processor 46 of the main server 14. The process steps of this portion of the process 1000 will be described below for purposes of this disclosure as being executed by the processor 46 of the main server 14. In some alternate embodiments, e.g., that may or may not include a main server 14, this portion of the process 1000 may alternatively be stored in the memory 38 (and/or data storage 40) of one or more of the local servers 32₁-32_{L} in the form of instructions executable by the processor 34 of the one or more local servers 32₁-32_{L}, stored in the memory of one of the point-of-sale systems 26₁-26_{M} within one or more of the brick-and-mortar enterprise stores or outlets in the form of instructions executable by a processor associated with any such point-of-sale system 26₁-26_{M} and/or stored in a memory and executable by a processor of another system external to or supplemental to the system 10 illustrated FIG. 1.

Another portion of the process 1000, i.e., the portion between the left-most vertical line and the right-most vertical line in FIG. 10, and centered under the heading “Cloud Server,” illustratively represents one or more software applications executed by a processor 60 of the cloud server 58. In one embodiment, this portion of the process 1000 is or includes the ready time module 332 stored in the memory 64 (and/or data storage 66) of the cloud server 58 in the form of instructions executable by the processor 60 of the cloud server 58 (see FIG. 3). The process steps of this portion of the process 1000 will be described below for purposes of this disclosure as being executed by the processor 60 of the cloud server 58.

Still another portion of the process 1000, i.e., the portion to the right of the right-most vertical line in FIG. 10, and centered under the heading “MCD,” illustratively represents one or more software applications executed by the processor of a patient's mobile communication device 18, i.e., one of the mobile communication devices 18₁-18_{J} associated with a patient of the retail pharmacy enterprise 12. In one embodiment, this portion of the process 1000 is or includes the web browser 408 stored in the memory 404 (and/or the data storage 410) of the patient's mobile communication device 18 (see FIG. 4) in the form of instructions executable by the processor 400 of the patient's mobile communication device 18. The process steps of this portion of the process 1000 will be described below for purposes of this disclosure as being executed by the processor 400 of the patient's mobile communication device 18.

It will further be understood that portions of the process 1000 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 1000 illustrated in FIG. 10 starts in step 1002, in which the cloud server 58 (e.g., the processor 46 of the main server 14) is operable to determine whether the status of a prescription transaction is “ready after time” or otherwise eligible to be updated by the patient. For example, the cloud server 58 may identify data payloads or other entries in the express cart collection 304 that have a status indicating that the prescription is predicted to be ready at a certain time. Records that are already indicated as completed or that do not include a ready time may not be selected. In step 1004, the cloud server 58 determines whether any prescription records with a status of “ready after time” have been identified. If not, the process 1000 branches to block 1006, in which the update ready time process is completed. If prescription records are identified, the process 1000 advances to step 1008.

In step 1008, the cloud server 58 provides a request new time/cancel interface to the MCD 18. The cloud server 58 may provide the request new time interface as a PWA, a web page, or other interface. For example, the cloud server 58 may provide a link or other user interface element to access the request new time interface in a shopping cart interface as described above. In response to selection of that link, the cloud server 58 may provide one or more additional user interfaces elements to allow the user to select a new ready time. The cloud server 58 may use a rules engine or other logic to identify available ready times.

In step 1010, the MCD 18 displays the request new time/cancel interface received from the cloud server 58. The MCD 18 may display the interface using a native web browser of the MCD 18. The interface may allow the user to select a new ready time for one or more prescriptions (e.g., “today after 5:00 p.m.,” “tomorrow,” or another requested time), to cancel one or more prescriptions, or to otherwise modify one or more prescriptions. In step 1012, the MCD 18 receives a user selection such as a requested time change or a cancellation. The MCD 18 provides a response including the user selection to the cloud server 58.

In step 1012, the cloud server 58 updates one or more prescription data entries based on the change request received from the MCD 18. The cloud server 58 may, for example, update corresponding entries in the express cart collection 304. The cloud server 58 may also send the change request to the main server 14, and in step 1016 the main server 14 may update one or more prescription transaction records based on the change request. For example, the main server 14 may update one or more records in the EPS 202 or other data system maintained by the main server 14. The main server 14 may send one or more notifications or other updates to the retail outlet 22 associated with the prescription transaction. After updating the main server 14, the process 1000 is completed.

### Drug Recall Notification Process

Referring now to FIG. 11, a simplified flow diagram is shown depicting an embodiment of a process 1100 for drug recall notification with enhanced messaging. As indicated by the framework of the process 1100 illustrated in FIG. 11, a portion of the process 1100, i.e., the portion to the left of the left-most vertical line and centered under the heading “Main Server,” illustratively represents one or more software applications executed by the processor 46 of the main server 14. In one embodiment, this portion of the process 1100 is or includes one or more of the modules stored in the EPS access module 206 (see FIG. 2) in the form of instructions executable by the processor 46 of the main server 14. The process steps of this portion of the process 1100 will be described below for purposes of this disclosure as being executed by the processor 46 of the main server 14. In some alternate embodiments, e.g., that may or may not include a main server 14, this portion of the process 1100 may alternatively be stored in the memory 38 (and/or data storage 40) of one or more of the local servers 32₁-32_{L} in the form of instructions executable by the processor 34 of the one or more local servers 32₁-32_{L}, stored in the memory of one of the point-of-sale systems 26₁-26_{M} within one or more of the brick-and-mortar enterprise stores or outlets in the form of instructions executable by a processor associated with any such point-of-sale system 26₁-26_{M} and/or stored in a memory and executable by a processor of another system external to or supplemental to the system 10 illustrated FIG. 1.

Another portion of the process 1100, i.e., the portion between the left-most vertical line and the right-most vertical line in FIG. 11, and centered under the heading “Cloud Server,” illustratively represents one or more software applications executed by a processor 60 of the cloud server 58. In one embodiment, this portion of the process 1100 is or includes the communication engine module 322 and the drug recall module 334 stored in the memory 64 (and/or data storage 66) of the cloud server 58 in the form of instructions executable by the processor 60 of the cloud server 58 (see FIG. 3). The process steps of this portion of the process 1100 will be described below for purposes of this disclosure as being executed by the processor 60 of the cloud server 58.

Still another portion of the process 1100, i.e., the portion to the right of the right-most vertical line in FIG. 11, and centered under the heading “MCD,” illustratively represents one or more software applications executed by the processor of a patient's mobile communication device 18, i.e., one of the mobile communication devices 18₁-18_{J} associated with a patient of the retail pharmacy enterprise 12. In one embodiment, this portion of the process 1100 is or includes the messaging application 406 and the web browser 408 stored in the memory 404 (and/or the data storage 410) of the patient's mobile communication device 18 (see FIG. 4) in the form of instructions executable by the processor 400 of the patient's mobile communication device 18. The process steps of this portion of the process 1100 will be described below for purposes of this disclosure as being executed by the processor 400 of the patient's mobile communication device 18.

It will further be understood that portions of the process 1100 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 1100 illustrated in FIG. 11 starts in step 1102, in which the main server 14 (e.g., the processor 46 of the main server 14) is operable to batch load drug recall information. The main server 14 may provide a data file or other data to the cloud server 58 that identifies drugs and/or patients affected by a drug recall. The drug recall information file may be extracted from one or more other databases or data collections. The drug recall information may be provided to the cloud server 58 using a web portal or other interface provided by the cloud server 58, and the cloud server 58 may store the drug recall information in bulk storage such as a “blob” storage 320 format.

In step 1104, the cloud server 58 (e.g., the processor 60 of the cloud server 58) processes the batch load drug recall information and stores associated drug recall documents. The cloud server 58 may, for example, ingest drug recall information from bulk storage and store the drug recall information as a data payload in a drug recall collection 310 or other data table. The cloud server 58 may store associated recall documents (e.g., PDF documents or other document representations) in blob storage 320. The drug recall collection 310 data payloads may include references or other links to the associated recall documents in blob storage 320.

In step 1106, the cloud server 58 provides the drug recall portal 346 to the main server 14. The recall portal 346 may be embodied as a web site or other portal that allows pharmacy users to view and manage recall status, including identifying whether patients have been notified of the drug recall, whether patients have acknowledged receiving the notification, and other information. The recall portal 346 may allow pharmacy users to filter recall information by physical retail outlet 22 or otherwise analyze recall data. In step 1108, the main server 14 may access the recall portal 346 provided by the cloud server 58. Additionally or alternatively, the recall portal 346 may be accessed by pharmacy users at a particular retail outlet 22, for example using one or more local hub servers 32, POS systems 26, or other devices. In addition to viewing recall data, pharmacy users may update recall information using the drug recall portal 346. For example, a pharmacy user may manually update drug recall status in response to providing drug recall information to a patient in person at the retail outlet 22 or otherwise providing the drug recall information.

After providing the recall portal 346, in step 1110 the cloud server 58 sends an enhanced message including a link to a drug recall progressive web application (PWA) to the MCD 18 associated with the patient. The cloud server 58 may send the enhanced message in response to a data payload in the drug recall collection matching one or more rules, such as rules determining that a patient is associated with one or more drugs that have been recalled. As described above, the enhanced message may be sent via SMS or other messaging service to a phone number associated with the patient. Also as indicated above, the enhanced message includes a link such as a URI or URL that identifies the drug recall PWA provided by the cloud server 58. In some embodiments, the PWA link may also include a unique identifier generated by the cloud server 58 and associated with the particular drug recall payload. As described further below, this unique identifier may be used by the cloud server 58 to associate PWA interactions with the MCD 18 with a particular drug recall, for example by referencing one or more records in the drug recall collection 310.

In step 1112, the MCD 18 associated with the patient receives and displays the enhanced message including the PWA link. The enhanced message may be displayed, for example, using the native messaging application 406 provided by the MCD 18. Thus, the enhanced message may be displayed and/or otherwise accessed without requiring the patient to download or install a custom application on the MCD 18. In step 1114, the MCD 18 receives a user selection of the drug recall PWA link. For example, the patient may tap, click, or otherwise select the PWA link displayed in the enhanced message. The MCD 18 may send a request for the URI or other resource identified by the PWA link to the cloud server 58.

In steps 1116 and 1118, the cloud server 58 and the MCD 18 cooperate to perform a verification process to verify the identity of the patient. For example, the cloud server 58 may send a challenge request to the MCD 18 that requests a date of birth or other verifying information associated with the patient. In response, the MCD 18 may send a response including a user selection of the date of birth or other verifying information. To verify the patient, the cloud server 58 and the MCD 18 may perform the verification process 700 illustrated in FIG. 7 and described above.

After verifying the patient, in step 1120, the cloud server 58 provides drug recall information to the MCD 18. The drug recall information may include a link to and/or a copy of the document(s) associated with the drug recall that are stored in blob storage 320 by the cloud server 58. The drug recall information may also include a checkbox or other user interface element usable by the patient to acknowledge receipt of the drug recall information. The cloud server 58 may provide the drug recall information as part of a PWA, a web page, or other interface.

In step 1122, the MCD 18 displays the drug recall information. The MCD 18 may also retrieve and display one or more documents associated with the drug recall information from the cloud server 58. In step 1124, the MCD 18 receives a user acknowledgement rom a user of the MCD 18. For example, the MCD 18 may receive a user selection of a checkbox or other user interface element acknowledging that the user received the drug recall information. The MCD 18 sends a response to the cloud server 58 including this user acknowledgment.

In step 1126, the cloud server 58 updates the drug recall portal 346 with the user acknowledgment. The cloud server 58 may, for example, update one or more entries in the drug recall collection 310 to set an acknowledgment flag and a date indicating that the patient acknowledged receipt of the drug recall information. Pharmacy users may access updated information provided by the drug recall portal 346 using the main server 14 and/or other devices. Thus, pharmacy users may use the drug recall portal 346 to track the status of each drug recall notification. After a predetermined amount of time (e.g., 72 hours), the drug recall portal 346 may be used to generate a mailing list or otherwise contact patients who have not yet acknowledged receipt of drug recall information.

### Prescription Refill Reminder Process

Referring now to FIG. 12, a simplified flow diagram is shown depicting an embodiment of a process 1200 for prescription refill reminders with enhanced messaging. As indicated by the framework of the process 1200 illustrated in FIG. 12, a portion of the process 1200, i.e., the portion to the left of the left-most vertical line and centered under the heading “Main Server,” illustratively represents one or more software applications executed by the processor 46 of the main server 14. In one embodiment, this portion of the process 1200 is or includes one or more of the modules stored in the EPS access module 206 (see FIG. 2) in the form of instructions executable by the processor 46 of the main server 14. The process steps of this portion of the process 1200 will be described below for purposes of this disclosure as being executed by the processor 46 of the main server 14. In some alternate embodiments, e.g., that may or may not include a main server 14, this portion of the process 1200 may alternatively be stored in the memory 38 (and/or data storage 40) of one or more of the local servers 32₁-32_{L} in the form of instructions executable by the processor 34 of the one or more local servers 32₁-32_{L}, stored in the memory of one of the point-of-sale systems 26₁-26_{M} within one or more of the brick-and-mortar enterprise stores or outlets in the form of instructions executable by a processor associated with any such point-of-sale system 26₁-26_{M} and/or stored in a memory and executable by a processor of another system external to or supplemental to the system 10 illustrated FIG. 1.

Another portion of the process 1200, i.e., the portion between the left-most vertical line and the right-most vertical line in FIG. 12, and centered under the heading “Cloud Server,” illustratively represents one or more software applications executed by a processor 60 of the cloud server 58. In one embodiment, this portion of the process 1200 is or includes the communication engine module 322 and the refill reminder module 336 stored in the memory 64 (and/or data storage 66) of the cloud server 58 in the form of instructions executable by the processor 60 of the cloud server 58 (see FIG. 3). The process steps of this portion of the process 1200 will be described below for purposes of this disclosure as being executed by the processor 60 of the cloud server 58.

Still another portion of the process 1200, i.e., the portion to the right of the right-most vertical line in FIG. 11, and centered under the heading “MCD,” illustratively represents one or more software applications executed by the processor of a patient's mobile communication device 18, i.e., one of the mobile communication devices 18₁-18_{J} associated with a patient of the retail pharmacy enterprise 12. In one embodiment, this portion of the process 1200 is or includes the messaging application 406 and the web browser 408 stored in the memory 404 (and/or the data storage 410) of the patient's mobile communication device 18 (see FIG. 4) in the form of instructions executable by the processor 400 of the patient's mobile communication device 18. The process steps of this portion of the process 1200 will be described below for purposes of this disclosure as being executed by the processor 400 of the patient's mobile communication device 18.

It will further be understood that portions of the process 1200 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 1200 illustrated in FIG. 12 starts in step 1202, in which the main server 14 (e.g., the processor 46 of the main server 14) is operable to batch load prescription refill information. To load the refill information, the main server 14 may extract information from an enterprise pharmacy system (EPS) 202 for prescriptions that are overdue for refill or are likely to be refilled soon. For example, the main server 14 may identify prescription records that have a refill date (e.g., the date the prescription was picked up plus the days supply of that prescription) that is a predetermined amount of time in the future (e.g., four days in the future). The prescription refill information may be provided to the cloud server 58 in a batch process. The cloud server 58 may load the prescription refill information into the batch outreach collection 308 or other data store maintained by the cloud server 58.

In step 1204, the cloud server 58 (e.g., the processor 60 of the cloud server 58) processes the batch load prescription refill information and sends an enhanced message including a link to a prescription refill progressive web application (PWA) to the MCD 18 associated with the patient. The cloud server 58 may send the enhanced message in response to a data payload in the batch outreach collection 308 matching one or more rules, such as rules determining that a patient is associated with prescriptions that are expected to be refilled. As described above, the enhanced message may be sent via SMS or other messaging service to a phone number associated with the patient. Also as indicated above, the enhanced message includes a link such as a URI or URL that identifies the refill reminder PWA provided by the cloud server 58. In some embodiments, the PWA link may also include a unique identifier generated by the cloud server 58 and associated with the particular recall reminder payload. As described further below, this unique identifier may be used by the cloud server 58 to associate PWA interactions with the MCD 18 with a particular recall reminder, for example by referencing one or more records in the batch outreach collection 308.

In step 1206, the MCD 18 associated with the patient receives and displays the enhanced message including the PWA link. The enhanced message may be displayed, for example, using the native messaging application 406 provided by the MCD 18. Thus, the enhanced message may be displayed and/or otherwise accessed without requiring the patient to download or install a custom application on the MCD 18. In step 1208, the MCD 18 receives a user selection of the prescription refill PWA link. For example, the patient may tap, click, or otherwise select the PWA link displayed in the enhanced message. The MCD 18 may send a request for the URI or other resource identified by the PWA link to the cloud server 58.

In steps 1210 and 1212, the cloud server 58 and the MCD 18 cooperate to perform a verification process to verify the identity of the patient. For example, the cloud server 58 may send a challenge request to the MCD 18 that requests a date of birth or other verifying information associated with the patient. In response, the MCD 18 may send a response including a user selection of the date of birth or other verifying information. To verify the patient, the cloud server 58 and the MCD 18 may perform the verification process 700 illustrated in FIG. 7 and described above.

After verifying the patient, in step 1214, the cloud server 58 provides an available prescription refill interface to the MCD 18. The available prescription refill interface may include information for one or more prescriptions available for refill such as drug name, refill due date, or other information. The available prescription refill interface may also include checkboxes or other user interface elements that allow a user to select prescriptions to refill. In some embodiments, the available prescription refill interface may include links or other user interface elements to provide additional information about each prescription, similar to the additional prescription details described above in connection with step 810 of FIG. 8. The cloud server 58 may provide the available prescription refill as part of a PWA, a web page, or other interface to the MCD 18.

In step 1216, the MCD 18 displays the prescription refill interface and receives a prescription refill user selection. The prescription refill interface may be displayed using a native web browser of the MCD 18. The user may select one or more prescriptions to refill using associated checkboxes or other user interface elements provided by the prescription refill interface. The MCD 18 sends a response to the cloud server 58 including the prescription refill user selection.

In step 1218, the cloud server 58 submits a prescription refill request to the main server 14 based on the user selection received from the MCD 18. The cloud server 58 may, for example, submit a request including one or more prescription identification numbers and one or more associated additional parameters. In some embodiments, those additional parameters may include whether the patient has authorized requesting additional refills from the associated provider, which may default to no authorization. In step 1220, the main server 14 processes the prescription refill request. For example, the main server 14 may submit the prescription refill request through an enterprise pharmacy system (EPS) 202 or other prescription management system. The main server 14 returns status information to the cloud server 58 indicating whether the prescription refill requests were successful.

In step 1222, the cloud server 58 provides prescription refill status information to the MCD 18. The status information may indicate whether or not each requested prescription refill was successfully submitted. The status information may be sent to the MCD 18 as a PWA, a web page, or other user interface. In step 1224, the MCD 18 displays the prescription status information received from the cloud server 58.

In step 1226, the cloud server 58 determines whether all requested prescription refills were successfully completed. If so, the process 1200 branches to step 1228, in which the process 1200 is completed. The submitted prescription refills may be processed and filled by the associated retail outlet 22 using the main server 24 and/or the associated EPS 202. As the refills are processed, the system 10 may perform additional processes, such as the update ready time process of FIG. 10 and/or the express checkout process of FIGS. 6, 8.

Referring back to step 1226, if at least some of the prescription refills were not successfully submitted, the process 1200 branches to step 1230, in which the cloud server 58 provides refill failure status information to the MCD 18. The refill failure status information may include information describing the cause of the refill failure, such as a lack of refills remaining in the prescription. The refill failure status may also include user interface elements requesting authorization to allow the pharmacy to contact the associated prescriber to request additional refills. Additionally or alternatively, the refill failure status may indicate that the prescriber has requested the patient to contact the prescriber directly to obtain additional refills. For other refill failure causes, the status information may include a telephone number or other contact information for the retail outlet 22 associated with the prescription refill. The failure status information may be sent to the MCD 18 as a PWA, a web page, or other user interface.

In step 1232, the MCD 18 displays the failure status information received from the cloud server 58 and may receive a user selection indicating whether the patient authorizes the pharmacy to request additional refills from the prescriber. The MCD 18 sends a response to the cloud server 58 including the user selection. In step 1234, the cloud server 58 determines whether the user authorized requesting additional refills from the prescriber. If not, the process 1200 branches to step 1238, in which the process 1200 is completed. If the user authorizes additional refills, the process 1200 branches to step 1236, in which the main server 14 processes the prescription refill request with a parameter indicating that requesting additional refills is authorized. For example, the main server 14 may submit the prescription refill request through the EPS 202 or other prescription management system as described above. The main server 14 returns status information to the cloud server 58 indicating whether the prescription refill requests were successful, and the process loops back to step 1222 to provide refill status as described above.

### Prescription Refill Request Process

Referring now to FIG. 13, a simplified flow diagram is shown depicting an embodiment of a process 1300 for processing patient prescription refill requests with enhanced messaging. As indicated by the framework of the process 1300 illustrated in FIG. 13, a portion of the process 1300, i.e., the portion to the left of the left-most vertical line and centered under the heading “Main Server,” illustratively represents one or more software applications executed by the processor 46 of the main server 14. In one embodiment, this portion of the process 1300 is or includes one or more of the modules stored in the EPS access module 206 (see FIG. 2) in the form of instructions executable by the processor 46 of the main server 14. The process steps of this portion of the process 1300 will be described below for purposes of this disclosure as being executed by the processor 46 of the main server 14. In some alternate embodiments, e.g., that may or may not include a main server 14, this portion of the process 1300 may alternatively be stored in the memory 38 (and/or data storage 40) of one or more of the local servers 32₁-32_{L} in the form of instructions executable by the processor 34 of the one or more local servers 32₁-32_{L}, stored in the memory of one of the point-of-sale systems 26₁-26_{M} within one or more of the brick-and-mortar enterprise stores or outlets in the form of instructions executable by a processor associated with any such point-of-sale system 26₁-26_{M} and/or stored in a memory and executable by a processor of another system external to or supplemental to the system 10 illustrated FIG. 1.

Another portion of the process 1300, i.e., the portion between the left-most vertical line and the right-most vertical line in FIG. 13, and centered under the heading “Cloud Server,” illustratively represents one or more software applications executed by a processor 60 of the cloud server 58. In one embodiment, this portion of the process 1300 is or includes the communication engine module 322 and the refill reminder module 336 stored in the memory 64 (and/or data storage 66) of the cloud server 58 in the form of instructions executable by the processor 60 of the cloud server 58 (see FIG. 3). The process steps of this portion of the process 1300 will be described below for purposes of this disclosure as being executed by the processor 60 of the cloud server 58.

Still another portion of the process 1300, i.e., the portion to the right of the right-most vertical line in FIG. 13, and centered under the heading “MCD,” illustratively represents one or more software applications executed by the processor of a patient's mobile communication device 18, i.e., one of the mobile communication devices 18₁-18_{J} associated with a patient of the retail pharmacy enterprise 12. In one embodiment, this portion of the process 1300 is or includes the messaging application 406 and the web browser 408 stored in the memory 404 (and/or the data storage 410) of the patient's mobile communication device 18 (see FIG. 4) in the form of instructions executable by the processor 400 of the patient's mobile communication device 18. The process steps of this portion of the process 1300 will be described below for purposes of this disclosure as being executed by the processor 400 of the patient's mobile communication device 18.

It will further be understood that portions of the process 1300 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 1300 illustrated in FIG. 13 starts in step 1302, in which the MCD 18 (e.g., the processor 400 of the MCD 18) is operable to send a message to the cloud server 58 requesting a prescription refill. The message may be an SMS text message or other message, and may be sent in response to a user selection in the native messaging application 406 or other application executed by the MCD 18. The body of the text message may include a predetermined textual command such as “REFILL.”

In step 1304, the cloud server 58 (e.g., the processor 60 of the cloud server 58) receives the text message from the MCD 18 and initiates the refill request process. The cloud server 58 may record the phone number or other identifier associated with the text message. In step 1306 the cloud server 58 responds to the MCD 18 with an enhanced message including a link to a refill request progressive web application (PWA). The enhanced message may be sent via SMS or other messaging service to the phone number associated with the text message received in step 1304. Also as indicated above, the enhanced message includes a link such as a URI or URL that identifies the refill request PWA provided by the cloud server 58.

In step 1308, the MCD 18 associated with the patient receives and displays the enhanced message including the PWA link. The enhanced message may be displayed, for example, using the native messaging application 406 provided by the MCD 18. Thus, the enhanced message may be displayed and/or otherwise accessed without requiring the patient to download or install a custom application on the MCD 18. In step 1310, the MCD 18 receives a user selection of the refill request PWA link. For example, the patient may tap, click, or otherwise select the PWA link displayed in the enhanced message. The MCD 18 may send a request for the URI or other resource identified by the PWA link to the cloud server 58.

In step 1312, the cloud server 58 provides a prescription refill request interface to the MCD 18. The prescription refill request interface may request a prescription number or other identifier associated with the prescription to be refilled. In some embodiments, the prescription refill interface may also include a store finder interface that may be used to identify a particular retail outlet 22, for example based on address or other geographic location. The cloud server 58 may provide the available prescription refill as part of a PWA, a web page, or other interface to the MCD 18.

In step 1314, the MCD 18 displays the prescription refill request interface and receives a prescription refill user selection. The prescription refill interface may be displayed using a native web browser of the MCD 18. The user may provide the prescription number or other identifier associated with the prescription requested to be refilled. In some embodiments, the user may also provide a selection of a retail outlet 22 using the store finder interface provided by the cloud server 58. The MCD 18 sends a response to the cloud server 58 including the prescription refill user selection.

In step 1316, the cloud server 58 parses the prescription number provided by the user selection and determines a store identifier associated with the physical retail outlet 22. In some embodiments, the store identifier may be embedded or otherwise encoded in the prescription number. Additionally or alternatively, the store identifier may be determined separately, for example using a store identifier lookup table.

After identifying the requested prescription number and store identifier, in step 1318 the cloud server 58 looks up the prescription associated with the refill request. The cloud server 58 submits a prescription refill request to the main server 14 based on the prescription number received from the MCD 18. The cloud server 58 may, for example, submit a request including one or more prescription identification numbers and one or more associated additional parameters. In some embodiments, those additional parameters may include whether the patient has authorized requesting additional refills from the associated provider, which may default to no authorization. In step 1320, the main server 14 processes the prescription refill request. For example, the main server 14 may submit the prescription refill request through an enterprise pharmacy system (EPS) 202 or other prescription management system. The main server 14 returns status information to the cloud server 58 indicating whether the prescription refill request was successful.

In step 1322, the cloud server 58 provides prescription refill status information to the MCD 18. The status information may indicate whether or not each requested prescription refill was successfully submitted. The status information may be sent to the MCD 18 as a PWA, a web page, or other user interface. In step 1324, the MCD 18 displays the prescription status information received from the cloud server 58.

In step 1326, the cloud server 58 determines whether all requested prescription refills were successfully completed. If so, the process 1300 branches to step 1328, in which the process 1300 is completed. The submitted prescription refills may be processed and filled by the associated retail outlet 22 using the main server 24 and/or the associated EPS 202. As the refills are processed, the system 10 may perform additional processes, such as the update ready time process of FIG. 10 and/or the express checkout process of FIGS. 6, 8.

Referring back to step 1326, if at least some of the prescription refills were not successfully submitted, the process 1300 branches to step 1330, in which the cloud server 58 provides refill failure status information to the MCD 18. The refill failure status information may include information describing the cause of the refill failure, such as a lack of refills remaining in the prescription. The refill failure status may also include user interface elements requesting authorization to allow the pharmacy to contact the associated prescriber to request additional refills. Additionally or alternatively, the refill failure status may indicate that the prescriber has requested the patient to contact the prescriber directly to obtain additional refills. For other refill failure causes, the status information may include a telephone number or other contact information for the retail outlet 22 associated with the prescription refill. The failure status information may be sent to the MCD 18 as a PWA, a web page, or other user interface.

In step 1332, the MCD 18 displays the failure status information received from the cloud server 58 and may receive a user selection indicating whether the patient authorizes the pharmacy to request additional refills from the prescriber. The MCD 18 sends a response to the cloud server 58 including the user selection. In step 1334, the cloud server 58 determines whether the user authorized requesting additional refills from the prescriber. If not, the process 1300 branches to step 1338, in which the process 1300 is completed. If the user authorizes additional refills, the process 1300 branches to step 1336, in which the main server 14 processes the prescription refill request with a parameter indicating that requesting additional refills is authorized. For example, the main server 14 may submit the prescription refill request through the EPS 202 or other prescription management system as described above. The main server 14 returns status information to the cloud server 58 indicating whether the prescription refill requests were successful, and the process loops back to step 1322 to provide refill status as described above.

### Optional Pin Verification Process

Referring now to FIG. 14, a simplified flow diagram is shown of an embodiment 1400 of the date of birth verification process with optional user-supplied personal identification number (PIN). The process 1400 may be executed by the cloud server 58 and MCD 18 in any steps used to verify identify of a patient, such as at steps 610, 612 of the process 600, steps 910, 912 of the process 900, steps 1116, 1118 of the process 1100, steps 1210, 1212 of the process 1200, or at other steps. As indicated by the framework of the process 1400 illustrated in FIG. 14, a portion of the process 1400, i.e., the portion to the left of the vertical line and centered under the heading “Cloud Server,” illustratively represents one or more software applications executed by a processor 60 of the cloud server 58. In one embodiment, this portion of the process 1400 is or includes PIN verification module 340 stored in the memory 64 (and/or data storage 66) of the cloud server 58 in the form of instructions executable by the processor 60 of the cloud server 58 (see FIG. 3). The process steps of this portion of the process 1400 will be described below for purposes of this disclosure as being executed by the processor 60 of the cloud server 58.

Still another portion of the process 1400, i.e., the portion to the right of the vertical line in FIG. 14, and centered under the heading “MCD,” illustratively represents one or more software applications executed by the processor of a patient's mobile communication device 18, i.e., one of the mobile communication devices 18₁-18j associated with a patient of the retail pharmacy enterprise 12. In one embodiment, this portion of the process 1400 is or includes the web browser 408 stored in the memory 404 (and/or the data storage 410) of the patient's mobile communication device 18 (see FIG. 4) in the form of instructions executable by the processor 400 of the patient's mobile communication device 18. The process steps of this portion of the process 1400 will be described below for purposes of this disclosure as being executed by the processor 400 of the patient's mobile communication device 18.

It will further be understood that portions of the process 1400 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 1400 illustrated in FIG. 14 starts in step 1402, in which the cloud server 58 (e.g., the processor 60 of the cloud server 58) is operable to determine whether a PIN has been previously associated with the patient. If so, the process 1400 advances to step 1434, described below. If a PIN has not been previously associated with the patient, the process advances to step 1404, in which the cloud server 58 requests date of birth from the patient. As discussed above, to request the patient's date of birth, the cloud server 58 may transmit a progressive web application (PWA), web page, or other challenge interface to the MCD 18 associated with the patient. The challenge interface may also include an option to specify a PIN instead of date of birth.

In step 1406, the MCD 18 receives a user selection from the user of the MCD 18. To receive the user selection, the MCD 18 may display the PWA, web page, or other challenge interface received from the cloud server 58. The challenge interface may include a date picker, text box, or other user interface elements configured to receive a date of birth from the user. As described above, the challenge interface may also include a link or other user interface element to request using a PIN instead of date of birth. The MCD 18 may display the interface using a native web browser.

In step 1408, the MCD 18 determines whether the user selected to add a PIN for future verification. If not, the process 1400 branches to step 1424, described below. If the user selected to add a PIN, the process 1400 advances to step 1410, in which the MCD 18 sends a response to the cloud server 58 that includes the date of birth user selection received from the user and the request to add a PIN.

In step 1412, the cloud server 58 compares the date of birth response received from the MCD 18 to a date of birth previously associated with the patient. For example, as described above, the patient may supply the date of birth when initially requesting the prescription fill or otherwise initiating the prescription transaction. The date of birth may be stored by the cloud server 58 in one or more data tables or other data storage. Additionally or alternatively, in some embodiments, the cloud server 58 may retrieve the date of birth from the main server 14 or other data source.

In step 1414, the cloud server 58 determines whether the birth date supplied by the MCD 18 matches the previously stored birth date. If not, the process 1400 branches to step 1416, in which the cloud server 58 indicates a verification error. In some embodiments, the process 1400 may be repeated one or more times in order to allow the user of the MCD 18 to attempt to repeat verification. After exceeding a predetermined number of attempts, (e.g., three attempts), the process 1400 may be locked for a predetermined time period (e.g., two hours), preventing further verification attempts.

Referring back to step 1414, if the birth date supplied by the MCD 18 matches the previously stored birth date, the process 1400 advances to step 1418, in which the cloud server 58 sends a set PIN interface to the MCD 18. In step 1420, the MCD 18 receives a user selection including the new PIN. The user may provide the new PIN using one or more numeric entry fields or other user interface elements. The MCD 18 sends a response to the cloud server 58 that includes the user selection with the new PIN. In step 1422, the cloud server 58 stores the PIN provided by the user for future use. After storing the PIN, the process 1400 loops back to step 1402, in which the cloud server 58 may verify the patient using the PIN.

Referring back to step 1408, if the user does not select to add a PIN (e.g., the user selects to verify using birth date), the process 1400 branches to step 1424. In step 1424, the MCD 18 sends a response to the cloud server 58 that includes the date of birth user selection received from the user.

In step 1426, the cloud server 58 compares the date of birth response received from the MCD 18 to a date of birth previously associated with the patient. For example, as described above, the patient may supply the date of birth when initially requesting the prescription fill or otherwise initiating the prescription transaction. The date of birth may be stored by the cloud server 58 in one or more data tables or other data storage. Additionally or alternatively, in some embodiments, the cloud server 58 may retrieve the date of birth from the main server 14 or other data source.

In step 1428, the cloud server 58 determines whether the birth date supplied by the MCD 18 matches the previously stored birth date. If not, the process 1400 branches to step 1430, in which the cloud server 58 indicates a verification error. As described above, the process 1400 may be repeated multiple times, and after exceeding a predetermined number of unsuccessful verification attempts, the process 1400 may be locked.

Referring back to step 1428, if the birth date supplied by the MCD 18 matches the previously stored birth date, the process 1400 advances to step 1432, in which the patient is successfully verified. After successful verification, the system 10 may proceed with the pending PWA process or other process. For example, as described above, after successful verification, the process 600 may continue executing in step 614 of the process 600, in step 914 of the process 900, in step 1120 of the process 1100, in step 1214 of the process 1200, or otherwise continue executing.

Referring back to step 1402, if a user PIN has previously been associated with the patient, the process 1400 advances to step 1434, in which the cloud server 58 requests the PIN from the patient. To request the PIN, the cloud server 58 may transmit a progressive web application (PWA), web page, or other challenge interface to the MCD 18 associated with the patient.

In step 1436, the MCD 18 receives a user selection of the PIN from the user of the MCD 18. To receive the user selection, the MCD 18 may display the PWA, web page, or other challenge interface received from the cloud server 58. The challenge interface may include one or more numeric entry fields or other user interface elements configured to receive the PIN from the user. The MCD 18 may display the interface using a native web browser. The MCD 18 sends a response to the cloud server 58 that includes the PIN user selection received from the user.

In step 1438, the cloud server 58 compares the PIN response received from the MCD 18 to the PIN previously associated with the patient as described above. In step 1440, the cloud server 58 determines whether the PIN supplied by the MCD 18 matches the previously stored PIN. If not, the process 1400 branches to step 1442, in which the cloud server 58 indicates a verification error. As described above, the process 1400 may be repeated multiple times, and after exceeding a predetermined number of unsuccessful verification attempts, the process 1400 may be locked.

Referring back to step 1440, if the PIN supplied by the MCD 18 matches the previously stored PIN, the process 1400 advances to step 1444, in which the patient is successfully verified. After successful verification, the system 10 may proceed with the pending PWA process or other process. For example, as described above, after successful verification, the process 600 may continue executing in step 614 of the process 600, in step 914 of the process 900, in step 1120 of the process 1100, in step 1214 of the process 1200, or otherwise continue executing.

### Immunization Request Process

Referring now to FIG. 15, a simplified flow diagram is shown of an embodiment 1500 of an immunization request process with enhanced messaging. As indicated by the framework of the process 1500 illustrated in FIG. 15, a portion of the process 1500, i.e., the portion to the left of the vertical line and centered under the heading “Cloud Server,” illustratively represents one or more software applications executed by a processor 60 of the cloud server 58. In one embodiment, this portion of the process 1500 is or includes communication engine module 322 and the immunization module 342 stored in the memory 64 (and/or data storage 66) of the cloud server 58 in the form of instructions executable by the processor 60 of the cloud server 58 (see FIG. 3). The process steps of this portion of the process 1500 will be described below for purposes of this disclosure as being executed by the processor 60 of the cloud server 58.

Still another portion of the process 1500, i.e., the portion to the right of the vertical line in FIG. 15, and centered under the heading “MCD,” illustratively represents one or more software applications executed by the processor of a patient's mobile communication device 18, i.e., one of the mobile communication devices 18₁-18j associated with a patient of the retail pharmacy enterprise 12. In one embodiment, this portion of the process 1500 is or includes the messaging application 406 and the web browser 408 stored in the memory 404 (and/or the data storage 410) of the patient's mobile communication device 18 (see FIG. 4) in the form of instructions executable by the processor 400 of the patient's mobile communication device 18. The process steps of this portion of the process 1500 will be described below for purposes of this disclosure as being executed by the processor 400 of the patient's mobile communication device 18.

It will further be understood that portions of the process 1500 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 1500 illustrated in FIG. 15 starts in step 1502, in which the MCD 18 (e.g., the processor 400 of the MCD 18) is operable to send a message to the cloud server 58 requesting an immunization appointment. The message may be an SMS text message or other message, and may be sent in response to a user selection in the native messaging application 406 or other application executed by the MCD 18. The body of the text message may include a predetermined textual command such as “FLU.”

In step 1504, the cloud server 58 (e.g., the processor 60 of the cloud server 58) receives the text message from the MCD 18 and initiates the immunization request process. The cloud server 58 responds to the MCD 18 with an enhanced message including a link to an immunization request progressive web application (PWA). The enhanced message may be sent via SMS or other messaging service to the phone number associated with the text message received from the MCD 18. Also as indicated above, the enhanced message includes a link such as a URI or URL that identifies the immunization request PWA provided by the cloud server 58.

In step 1506, the MCD 18 associated with the patient receives and displays the enhanced message including the PWA link. The enhanced message may be displayed, for example, using the native messaging application 406 provided by the MCD 18. Thus, the enhanced message may be displayed and/or otherwise accessed without requiring the patient to download or install a custom application on the MCD 18. In step 1508, the MCD 18 receives a user selection of the immunization request PWA link. For example, the patient may tap, click, or otherwise select the PWA link displayed in the enhanced message. The MCD 18 may send a request for the URI or other resource identified by the PWA link to the cloud server 58.

In step 1510, the cloud server 58 provides an immunization request interface to the MCD 18. The immunization request interface may request from the MCD 18 multiple items of information usable to schedule an immunization appointment. For example, the immunization request interface may request a prescription number or other identifier associated with the prescription to be refilled. In some embodiments, the prescription refill interface may also include a store finder interface that may be used to identify a particular retail outlet 22, for example based on address or other geographic location. The cloud server 58 may provide the available prescription refill as part of a PWA, a web page, or other interface to the MCD 18.

In step 1512, the MCD 18 receives a user selection of a store location. The MCD 18 may receive the user selection in response to displaying a store finder interface received from the cloud server 58. The user may identify a particular retail outlet 22, for example using address or other geographic information. The MCD 18 sends a response including the user selection of the store location to the cloud server 58. In step 1514, the cloud server 58 looks up a store location based on the user selection. The cloud server 58 may, for example, look up a store number or other identification in a store lookup table.

In step 1516, the cloud server 58 provides a patient identification interface to the MCD 18. The patient identification interface may include fields for providing one or more items of identifying information relating to a patient, such as last name, birthdate, and postal code (e.g., ZIP code). In step 1518, the MCD 18 receives a user selection including patient identification information. The MCD 18 sends a response to the cloud server 58 including the user selection.

In step 1520, the cloud server 58 verifies the patient information provided by the MCD 18. The cloud server 58 may, for example, look up patient records in a patient lookup table 318 that match the identifying information provided by the MCD 18. The patient lookup table 318 may be initially populated with patient data provided by the main server 14 or otherwise extracted from the enterprise pharmacy system (EPS) 202. Thus, the cloud server 58 may verify the identity of the patient without requiring the patient to create an online account. The cloud server 58 may also verify additional information associated with the patient, such as whether the patient has opted in to enhanced messaging as described above in connection with FIG. 9. If the patient information is not successfully verified (not shown), the cloud server 58 may allow the patient to make multiple verification attempts and/or generate an error message. The error message may include contact information for the selected retail outlet 22, which may allow the patient to receive assistance.

After verifying the identifying information provided by the MCD 18, the process 1500 advances to step 1522, in which the cloud server 58 generates a two-factor authentication code. The two-factor authentication code may be embodied as a random numeric code or any other random code. The cloud server 58 sends the two-factor authentication code to a mobile phone number associated with the patient using a text messaging service.

In step 1524, the MCD 18 associated with the patient receives and displays the message including the two-factor authentication code. The two-factor authentication code may be displayed, for example, using the native messaging application 406 provided by the MCD 18. In step 1526, the MCD 18 receives a user selection including the two-factor authentication code. The user selection may be received, for example, using a PWA received from the cloud server 58 and executed by the native web browser of the MCD 18. Continuing that example, the user may manually input the two-factor authentication code received via text message into the PWA interface. The MCD 18 sends a response to the cloud server 58 that includes the user selection of the two-factor authentication code.

In step 1528, the cloud server 58 compares the user selection received from the MCD 18 to the two-factor authentication code determined as described above in connection with step 1522. In block 1530, the cloud server 58 determines whether the authentication codes match. If not, the process 1500 branches to step 1532, in which the cloud server 58 indicates an error has occurred. In response to an error, the cloud server 58 may allow multiple attempts for two-factor authentication. For example, the cloud server 58 may generate another two-factor authentication code, and/or the MCD 18 may allow the user to re-submit the two-factor authentication code. After a predetermined number of attempts, the cloud server 58 may lock the immunization request process. Referring back to step 1530, if the authentication codes match, the process 1500 advances to step 1534.

In step 1534, the cloud server 58 provides an immunization appointment message to the MCD 18. Before sending the message, the cloud server 58 may update the immunization collection 316, the patient lookup table 318, and/or or other data tables to indicate that an immunization request has been processed. The immunization appointment message may include a link to an immunization appointment PWA. In step 1536, the MCD 18 displays the immunization appointment message. One potential embodiment of a method for preparing for an immunization appointment is described below in connection with FIG. 16.

### Immunization Appointment Preparation Process

Referring now to FIG. 16, a simplified flow diagram is shown depicting an embodiment of a process 1600 for preparing for an immunization appointment with an immunization PWA. As indicated by the framework of the process 1600 illustrated in FIG. 16, a portion of the process 1600, i.e., the portion to the left of the left-most vertical line and centered under the heading “Main Server,” illustratively represents one or more software applications executed by the processor 46 of the main server 14. In one embodiment, this portion of the process 1600 is or includes one or more of the modules stored in the EPS access module 206 (see FIG. 2) in the form of instructions executable by the processor 46 of the main server 14. The process steps of this portion of the process 1600 will be described below for purposes of this disclosure as being executed by the processor 46 of the main server 14. In some alternate embodiments, e.g., that may or may not include a main server 14, this portion of the process 1600 may alternatively be stored in the memory 38 (and/or data storage 40) of one or more of the local servers 32₁-32_{L} in the form of instructions executable by the processor 34 of the one or more local servers 32₁-32_{L}, stored in the memory of one of the point-of-sale systems 26₁-26_{M} within one or more of the brick-and-mortar enterprise stores or outlets in the form of instructions executable by a processor associated with any such point-of-sale system 26₁-26_{M} and/or stored in a memory and executable by a processor of another system external to or supplemental to the system 10 illustrated FIG. 1.

Another portion of the process 1600, i.e., the portion between the left-most vertical line and the right-most vertical line in FIG. 16, and centered under the heading “Cloud Server,” illustratively represents one or more software applications executed by a processor 60 of the cloud server 58. In one embodiment, this portion of the process 1600 is or includes the communication engine module 322 and the immunization module 342 stored in the memory 64 (and/or data storage 66) of the cloud server 58 in the form of instructions executable by the processor 60 of the cloud server 58 (see FIG. 3). The process steps of this portion of the process 1600 will be described below for purposes of this disclosure as being executed by the processor 60 of the cloud server 58.

Still another portion of the process 1600, i.e., the portion to the right of the right-most vertical line in FIG. 16, and centered under the heading “MCD,” illustratively represents one or more software applications executed by the processor of a patient's mobile communication device 18, i.e., one of the mobile communication devices 18₁-18_{J} associated with a patient of the retail pharmacy enterprise 12. In one embodiment, this portion of the process 1600 is or includes the messaging application 406 and the web browser 408 stored in the memory 404 (and/or the data storage 410) of the patient's mobile communication device 18 (see FIG. 4) in the form of instructions executable by the processor 400 of the patient's mobile communication device 18. The process steps of this portion of the process 1600 will be described below for purposes of this disclosure as being executed by the processor 400 of the patient's mobile communication device 18.

It will further be understood that portions of the process 1600 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 1600 illustrated in FIG. 16 starts in step 1602, in which the main server 14 (e.g., the processor 46 of the main server 14) is operable to request an immunization message. The immunization message may be requested, for example, in response to successfully completing the immunization request process described above in connection with FIG. 15. As another example, the immunization message may be requested manually by a pharmacy user or other user of the main server 14, for example using the immunization portal 348 provided by the cloud server 58. Additionally or alternatively, although illustrated as being requested by the main server 14, it should be understood that in some embodiments, the immunization message may be requested by a different device, such as the cloud server 58.

In step 1604, the cloud server 58 (e.g., the processor 60 of the cloud server 58) sends an enhanced message including a link to an immunization progressive web application (PWA) to the MCD 18 associated with the patient. The cloud server 58 may send the enhanced message in response to a data payload matching one or more rules, such as rules determining that an immunization message has been requested. As described above, the enhanced message may be sent via SMS or other messaging service to a phone number associated with the patient. Also as indicated above, the enhanced message includes a link such as a URI or URL that identifies the immunization PWA provided by the cloud server 58.

In step 1606, the MCD 18 associated with the patient receives and displays the enhanced message including the PWA link. The enhanced message may be displayed, for example, using the native messaging application 406 provided by the MCD 18. Thus, the enhanced message may be displayed and/or otherwise accessed without requiring the patient to download or install a custom application on the MCD 18. In step 1608, the MCD 18 receives a user selection of the immunization PWA link. For example, the patient may tap, click, or otherwise select the PWA link displayed in the enhanced message. The MCD 18 may send a request for the URI or other resource identified by the PWA link to the cloud server 58.

In steps 1610 and 1612, the cloud server 58 and the MCD 18 cooperate to perform a verification process to verify the identity of the patient. For example, the cloud server 58 may send a challenge request to the MCD 18 that requests a date of birth or other verifying information associated with the patient. In response, the MCD 18 may send a response including a user selection of the date of birth or other verifying information. To verify the patient, the cloud server 58 and the MCD 18 may perform the verification process 700 illustrated in FIG. 7 and described above.

After verifying the patient, in step 1614, the cloud server 58 provides an immunization survey interface to the MCD 18. The survey interface includes one more user interface elements requesting information from the patient required before performing an immunization. For example, the survey may request the patient to respond whether the patient is feeling sick, whether the patient has ever had a serious reaction after receiving a vaccination, or other information. In step 1616, the MCD 18 receives a user selection from the user answering the survey questions. The MCD 18 sends a response to the cloud server 58 including the user selection. The cloud server 58 may store the user selection in the immunization collection 316 or other data storage.

In step 1618, the cloud server 58 provides a consent form to the MCD 18. The consent form may include required consent language and user interface controls to receive an indication of consent from the patient. In step 1620, the MCD 18 displays the consent form and receives a user selection from the patient. The user selection, such as input into a checkbox or other user interface element, indicates that the user consents to the vaccination. The MCD 18 sends a response to the cloud server 58 including the user selection.

In step 1622, the cloud server 58 requests a signature from the user. The cloud server 58 may send a signature interface to the MCD 18 or otherwise request the patient signature. In step 1624, the MCD 18 displays the signature interface and collects a patient signature from the user. The signature may be collected, for example, using a touch screen or other interface device of the MCD 18. After collecting the signature, the MCD 18 sends data indicative of the signature (e.g., one or more image files) to the cloud server 58.

In step 1626, the cloud server 58 generates a consent form including the language of the consent form provided in step 1618 and the signature collected in step 1622. The cloud server 58 stores the consent form. The consent form may be stored, for example, in the blob storage 320 or in a patient profile stored in an enterprise pharmacy system (EPS) 202 or other data source associated with the patient.

In step 1628, the cloud server 58 updates the immunization portal 348, for example by submitting updated vaccination information to the main server 14. In step 1630, the main server 14 updates a vaccination workflow. The main server 14 may store the consent form, prescription, and other data in one or more EPS 202 records. Updating the workflow allows the immunization to proceed at the associated retail outlet 22.

After performing the immunization at the retail outlet 22, in step 1632, the main server 14 completes the associated vaccination workflow. The vaccination may be marked as complete in the EPS 202 system, for example in response to one or more commands entered through the immunization portal 348. Additionally, information regarding the vaccination may be submitted to an associated prescriber and any associated state vaccination registries.

### Dynamic Two-Way Communication Process

Referring now to FIG. 17, a simplified flow diagram is shown depicting an embodiment of a process 1700 for dynamic two-way communications incorporating progressive web applications (PWAs). As indicated by the framework of the process 1700 illustrated in FIG. 17, a portion of the process 1700, i.e., the portion to the left of the left-most vertical line and centered under the heading “Main Server,” illustratively represents one or more software applications executed by the processor 46 of the main server 14. In one embodiment, this portion of the process 1700 is or includes one or more of the modules stored in the EPS access module 206 (see FIG. 2) in the form of instructions executable by the processor 46 of the main server 14. The process steps of this portion of the process 1700 will be described below for purposes of this disclosure as being executed by the processor 46 of the main server 14. In some alternate embodiments, e.g., that may or may not include a main server 14, this portion of the process 1700 may alternatively be stored in the memory 38 (and/or data storage 40) of one or more of the local servers 32₁-32_{L} in the form of instructions executable by the processor 34 of the one or more local servers 32₁-32_{L}, stored in the memory of one of the point-of-sale systems 26₁-26_{M} within one or more of the brick-and-mortar enterprise stores or outlets in the form of instructions executable by a processor associated with any such point-of-sale system 26₁-26_{M} and/or stored in a memory and executable by a processor of another system external to or supplemental to the system 10 illustrated FIG. 1.

Another portion of the process 1700, i.e., the portion between the left-most vertical line and the right-most vertical line in FIG. 17, and centered under the heading “Cloud Server,” illustratively represents one or more software applications executed by a processor 60 of the cloud server 58. In one embodiment, this portion of the process 1700 is or includes the communication engine 322 and the two-way interaction module 344 stored in the memory 64 (and/or data storage 66) of the cloud server 58 in the form of instructions executable by the processor 60 of the cloud server 58 (see FIG. 3). The process steps of this portion of the process 1700 will be described below for purposes of this disclosure as being executed by the processor 60 of the cloud server 58.

Still another portion of the process 1700, i.e., the portion to the right of the right-most vertical line in FIG. 17, and centered under the heading “MCD,” illustratively represents one or more software applications executed by the processor of a patient's mobile communication device 18, i.e., one of the mobile communication devices 18₁-18ⱼ associated with a patient of the retail pharmacy enterprise 12. In one embodiment, this portion of the process 1700 is or includes the messaging application 406 and the web browser 408 stored in the memory 404 (and/or the data storage 410) of the patient's mobile communication device 18 (see FIG. 4) in the form of instructions executable by the processor 400 of the patient's mobile communication device 18. The process steps of this portion of the process 1700 will be described below for purposes of this disclosure as being executed by the processor 400 of the patient's mobile communication device 18.

It will further be understood that portions of the process 1700 illustrated as being executed by one processor/device or one processor/server or one processor/system may alternatively be executed by a different processor/device or processor/server or processor/system in the system 10, and/or by two or more such processors in any one or combination of such devices, servers and/or systems, some examples of which are described above.

The process 1700 illustrated in FIG. 17 starts in step 1702, in which the main server 14 (e.g., the processor 46 of the main server 14) is operable to create or update an enterprise pharmacy system (EPS) 202 record. The EPS 202 may include a database, portal, and/or other systems used by the retail pharmacy enterprise 12 to manage workflow, track orders, manage patient relations, and perform other tasks. In the illustrative embodiment the main server 14 may create or update an EPS 202 record in response to a patient requesting to fill one or more prescriptions, for example at a retail location 22 or otherwise. Additionally or alternatively, in some embodiments the main server 14 may create or update an EPS 202 record on demand, for example in response to a command from a pharmacy user.

In step 1704, the main server 14 publishes an EPS 202 payload that includes data representing the created and/or updated EPS 202 record. The main server 14 may publish the EPS 202 payload periodically, responsively, or otherwise, and may use any appropriate technique to publish the EPS 202 payload. As shown, the published EPS 202 payload is received and processed by the cloud server 58. In the illustrative embodiment, the main server 14 may publish the EPS 202 payload using an EPS Outbound Patient Notifications (EOPN) service as one or more SOAP (i.e., Simple Object Access Protocol) messages to the cloud server 58. The cloud server 58 may store the payload and/or data derived from the payload in one or more data stores 302 managed by or accessible to the cloud server 58, such as the two-way collection 314.

In step 1706, the cloud server 58 (e.g., the processor 60 of the cloud server 58) matches the EPS 202 payload against multiple communication rules. Each of the communication rules may be embodied as any conditions, database queries, or other computer code that may be evaluated against the EPS 202 payload to determine whether the EPS 202 payload matches the communication rule. In some embodiments, each communication rule and/or a rules engine may be embodied as a function, lambda, or other code executed by the cloud server 58 using a function as a service (FaaS) platform or other “serverless” architecture.

In step 1708, the cloud server 58 builds an enhanced message from one or more templates associated with a communication rule that matches the EPS 202 payload. Each template may be associated with a different patient interaction or part of a different patient interaction. Each template may include static text or other content used to communicate with the patient as well data from the EPS 202 payload and/or other data stores 302 maintained by the cloud server 58 and/or the main server 14. As described above, the cloud server 58 may maintain templates for certain pre-configured messages or other interactions, such as the illustrative embodiments shown above in Table 1.

In step 1710, the cloud server 58 sends the enhanced message to the patient. The cloud server 58 sends the message via short message service (SMS) or other messaging service to a phone number associated with the EPS 202 payload (and, thus, the patient). For example, the cloud server 58 may send the message to a phone number provided by the patient when initially requesting the prescription fill. The enhanced message includes a link to a progressive web application (PWA). The link may be embodied as a uniform resource indicator (URI), uniform resource locator (URL), or other address that identifies a particular PWA or other web application resource provided by the cloud server 58. In some embodiments, the PWA may include a unique identifier associated with the particular patient, such as a random identification number generated by the cloud server 58. Illustratively, the link may identify a landing page or other entry point associated with a two-way communication PWA.

In step 1712, the MCD 18 associated with the patient receives and displays the enhanced message including the PWA link. The enhanced message may be displayed, for example, using the native messaging application 406 provided by the MCD 18. Thus, the enhanced message may be displayed and/or otherwise accessed without requiring the patient to download or install a custom application on the MCD 18. In step 1714, the MCD 18 receives a user selection of the PWA link. For example, the patient may tap, click, or otherwise select the PWA link displayed in the enhanced message. In response to the user selection, the MCD 18 may send a request to the cloud server 58 to access the PWA link.

In step 1716, the cloud server 58 looks up an interaction component for the current PWA state in the two-way configuration table 312. The interaction component may be one or more PWA templates, web pages, documents, or other application components that the cloud server 58 and the MCD 18 are to perform as part of the current PWA. For example, the interaction component may be embodied as a verify patient identity component, a read message component, a question component, a survey component, a review documents or links component, a signature capture component, or a complete screen component. The configuration table 312 may identify the appropriate interaction component based on the reason for patient interaction. The reason for interaction, in turn, may be determined based on current state of the data payload received from the main server 14, the two-way collection 314, or other data collections maintained by the cloud server 58. Thus, one or more interaction flows or journeys may be defined in the configuration table 316, and the cloud server 58 may select the appropriate interaction based on current state. Potential interaction flows may be configured for business processes including communication consent, add credit card, transfer follow-up, call back needed, PA submission, PA response, support program, service confirmation, welcome packet, privacy practice notice, transaction audit signature, HIPAA signature collect, Medicare open enrollment, or other processes. In particular, one or more of the processes described above, such as the immunization process 1600 shown in FIG. 16, may be implemented using dynamic two-way communication as described herein.

In step 1718, the cloud server 58 builds a PWA interface from one or more templates associated with the interaction component determined in step 1716. As described above, the PWA interface may be embodied as a web application including one or more web pages, scripts, or other resources to be executed or otherwise displayed by the MCD 18. Thus, the cloud server 58 dynamically identifies the interaction component and then dynamically assembles the associated PWA interface.

In step 1720, the cloud server 58 provides the PWA interface component to the MCD 18. In response, in step 1722 the MCD 18 displays the PWA interface component. For example, the MCD 18 may display the PWA interface in a native web browser provided by the MCD 18. Thus, the MCD 18 may also execute the dynamic PWA without requiring the user to download or install a custom application on the MCD 18. In some embodiments, after displaying the PWA interface component, the process 1700 loops back to step 1716, in which the cloud server 58 may look up additional interaction components. For example, for informational or read-only interaction components (e.g., a read message component, a review documents or links component, or a complete screen component), the process 1700 may loop back to step 1716 without receiving a user selection from the MCD 18.

In some embodiments, in step 1722, the MCD 18 may receive a user selection in response to displaying the PWA interface. Certain interaction components may include user interface elements configured to receive a user selection from the user of the MCD 18. For example, interactive components such as a verify patient identity component, a question component, a survey component, or a signature capture component may include user interface elements that capture one or more user selections. The MCD 18 responds to the cloud server 58 with a response that includes the user selections. In step 1726, the MCD 18 may update the PWA state based on the user selection. For example, the MCD 18 may update the data payload in the two-way collection 314 or another data collection based on the contents of the user selection. In some embodiments, in step 1728, the main server 14 may update an EPS 202 record based on data provided by the cloud server 58 (which, in turn, may be based on the user selection). After performing those updates, the process 1700 loops back to step 1716, in which the cloud server 58 may look up additional interaction components

While the disclosure has been illustrated and described in detail in the drawings and foregoing description, such an illustration and description is to be considered as exemplary and not restrictive in character, it being understood that only illustrative embodiments have been shown and described and that all changes and modifications consistent with the disclosure and recited claims are desired to be protected. For example, it will be understood that while several process steps in various sequences have been illustrated and described herein with respect to the processes 500, 600, 700, 800, 900, 1000, 1100, 1200, 1300, 1400, 1500, 1600 and 1700, any one or more such processes 500, 600, 700, 800, 900, 1000, 1100, 1200, 1300, 1400, 1500, 1600 and 1700 may alternatively include more, fewer and/or different steps, and that any such steps may be executed in different sequences from those illustrated and described, without departing from the scope of the concepts and techniques described herein.

In any of the foregoing embodiments, information may be transmitted, receive and/or processed by any one or combination of any system or device disclosed herein.

Further information relating to the structure and operation of the system(s) and method(s) disclosed herein can be found in the Appendix attached hereto, the contents of which form part of this disclosure.

What is claimed is:

1. A method for enhanced messaging with a patient of a retail pharmacy, the method comprising:
matching, by a cloud server, a data payload to one a plurality of communication rules, wherein each of the plurality of communication rules is associated with a message type, and wherein the data payload is indicative of a prescription transaction provided to the retail pharmacy for the patient;
creating, by the cloud server, a message based on the message type associated with the matching one of the plurality of communication rules, wherein the message includes an application address associated with the message type;
sending, by the cloud server, the message to a mobile communication device associated with the data payload and with the patient; and
in response to receipt by the cloud server of selection of the application address via patient interaction with the mobile communication device, executing, by the cloud server with the mobile communication device, a progressive web application associated with the application address via which the retail pharmacy interactively provides at least one retail pharmacy service to the patient.

2. The method of claim 1, further comprising:
sending, by the cloud server, a request for verifying information to the mobile communications device in response to selection of the application address via patient interaction with the mobile communication device;
receiving, by the cloud server, a patient selection indicative of the verifying information from the mobile communications device in response to sending the request; and
determining, by the cloud server, whether the patient selection matches an item of verifying information from the data payload.

3. The method of claim 2, wherein the verifying information comprises a date of birth of the patient.

4. The method of claim 2, further comprising executing, by the cloud server with the mobile communications device, the progressive web application upon determining that the patient selection matches the item of verifying information.

5. The method of claim 1, wherein executing the progressive web application comprises executing an express checkout shopping cart application via which the cloud server provides a prescription shopping cart interface to the mobile communication device for selection by the patient, via interaction by the patient with the mobile communication device, of one or more elements of a prescription transaction including a payment method.

6. The method of claim 5, wherein executing the express checkout shopping cart application comprises:
determining whether the data payload includes a patient selection to opt in to view details; and
masking private health information in response to determining that the data payload does not include the patient selection to opt in.

7. The method of claim 1, wherein executing the progressive web application comprises executing a view details enrollment application via which the patient provides patient information to the cloud server by interacting with the mobile communication device.

8. The method of claim 1, wherein executing the progressive web application comprises executing a drug recall application via which the cloud server sends at least one message to the mobile communication device informing the patient of drug recall information.

9. The method of claim 1, wherein executing the progressive web application comprises executing a prescription refill application via which the cloud server provides a prescription refill interface to the mobile communication device for selection by the patient, via interaction by the patient with the mobile communication device, of one or more elements of a prescription refill request.

10. The method of claim 1, wherein executing the progressive web application comprises executing an immunization application via which the cloud server provides an immunization request interface to the mobile communication device for selection by the patient, via interaction by the patient with the mobile communication device, of one or more elements for scheduling an immunization appointment with the retail pharmacy.

11. The method of claim 1, further comprising:
generating, by the cloud server, a progressive web application interface for the progressive web application from a plurality of application templates based on the data payload and in response to selection of the application address via patient interaction with the mobile communication device; and
transmitting, by the cloud server, the progressive web application interface to the mobile communications device.

12. The method of claim 11, further comprising:
receiving, by the cloud server, a patient selection from the mobile communications device in response to transmitting the progressive web application interface; and
updating, by the cloud server, the data payload based on the patient selection.

13. A cloud server for enhanced messaging with a patient of a retail pharmacy, the cloud server comprising a processor and a memory having instructions stored therein and executable by the processor to cause the processor to:
match a data payload to one of a plurality of communication rules, wherein each of the plurality of communication rules is associated with a message type, and wherein the data payload is indicative of a prescription transaction provided to the retail pharmacy for the patient;
create a message based on the message type associated with the matching one of the plurality of communication rules, wherein the message includes an application address associated with the message type;
send the message to a mobile communication device associated with the data payload and with the patient; and
in response to receipt of selection of the application address via patient interaction with the mobile communication device, execute with the mobile communication device a progressive web application associated with the application address via which the retail pharmacy interactively provides at least one retail pharmacy service to the patient.

14. The cloud server of claim 13, wherein the instructions stored in the memory include instructions executable by the processor to cause the processor to:
send a request for verifying information to the mobile communications device in response to selection of the application address via patient interaction with the mobile communication device;
receive a patient selection indicative of the verifying information from the mobile communications device in response to sending the request;
determine whether the patient selection matches an item of verifying information from the data payload; and
execute with the mobile communications device the progressive web application upon determining that the patient selection matches the item of verifying information.

15. The cloud server of claim 13, wherein the instructions stored in the memory include instructions executable by the processor to cause the processor to:
generate a progressive web application interface for the progressive web application from a plurality of application templates based on the data payload and in response to selection of the application address via patient interaction with the mobile communication device; and
transmit the progressive web application interface to the mobile communications device.

16. The cloud server of claim 13, wherein the progressive web application comprises at least one of:
an express checkout shopping cart application via which the cloud server provides a prescription shopping cart interface to the mobile communication device for selection by the patient, via interaction by the patient with the mobile communication device, of one or more elements of a prescription transaction including a payment method;
a view details enrollment application via which the patient provides patient information to the cloud server by interacting with the mobile communication device;
a drug recall application via which the cloud server sends at least one message to the mobile communication device informing the patient of drug recall information;
a prescription refill application via which the cloud server provides a prescription refill interface to the mobile communication device for selection by the patient, via interaction by the patient with the mobile communication device, of one or more elements of a prescription refill request; and
an immunization application via which the cloud server provides an immunization request interface to the mobile communication device for selection by the patient, via interaction by the patient with the mobile communication device, of one or more elements for scheduling an immunization appointment with the retail pharmacy.

17. A non-transitory machine-readable medium comprising a plurality of instructions executable by at least one processor to cause the at least one processor to:
match a data payload to one of a plurality of communication rules, wherein each of the plurality of communication rules is associated with a message type, and wherein the data payload is indicative of a prescription transaction provided to the retail pharmacy for the patient;
create a message based on the message type associated with the matching one of the plurality of communication rules, wherein the message includes an application address associated with the message type;
send the message to a mobile communication device associated with the data payload and with the patient; and
in response to receipt of selection of the application address via patient interaction with the mobile communication device, execute with the mobile communication device a progressive web application associated with the application address via which the retail pharmacy interactively provides at least one retail pharmacy service to the patient.

18. The non-transitory machine-readable medium of claim 17, wherein the plurality of instructions includes instructions executable by the at least one processor to cause the at least one processor to:
send a request for verifying information to the mobile communications device in response to selection of the application address via patient interaction with the mobile communication device;
receive a patient selection indicative of the verifying information from the mobile communications device in response to sending the request;
determine whether the patient selection matches an item of verifying information from the data payload; and
execute, with the mobile communications device, the progressive web application upon determining that the patient selection matches the item of verifying information.

19. The non-transitory machine-readable medium of claim 17, wherein the plurality of instructions includes instructions executable by the at least one processor to cause the at least one processor to:
generate a progressive web application interface for the progressive web application from a plurality of application templates based on the data payload and in response to selection of the application address via patient interaction with the mobile communication device; and
transmit the progressive web application interface to the mobile communications device.

20. The non-transitory machine-readable medium of claim 17, wherein the plurality of instructions includes instructions executable by the at least one processor to cause the at least one processor to execute the progressive web application by executing at least one of:
an express checkout shopping cart application to provide a prescription shopping cart interface to the mobile communication device for selection by the patient, via interaction by the patient with the mobile communication device, of one or more elements of a prescription transaction including a payment method;
a view details enrollment application via which the patient provides patient information to the cloud server by interacting with the mobile communication device;
a drug recall application via which the cloud server sends at least one message to the mobile communication device informing the patient of drug recall information;
a prescription refill application via which the cloud server provides a prescription refill interface to the mobile communication device for selection by the patient, via interaction by the patient with the mobile communication device, of one or more elements of a prescription refill request; and
an immunization application via which the cloud server provides an immunization request interface to the mobile communication device for selection by the patient, via interaction by the patient with the mobile communication device, of one or more elements for scheduling an immunization appointment with the retail pharmacy.
